(19) United States
(12) Patent Application Publication
(10) Pub. No.: US 2024/0164740 A1
(43) Pub. Date: May 23, 2024

(54) Modular Stethoscope

(71) Applicant: 3M INNOVATIVE PROPERTIES COMPANY, St. Paul, MN (US)

(72) Inventors: William Bedingham, Woodbury, MN (US); Daniel J. Rogers, Grant, MN (US)

(21) Appl. No.: 18/550,302

(22) PCT Filed: Mar. 17, 2022
(86) PCT No.: PCT/IB2022/052453

§ 371 Date: Sep. 13, 2023

Related U.S. Application Data
(60) Provisional application No. 63/200,722, filed on Mar. 24, 2021.

(51) Int. Cl. A61B 7/04

(52) U.S. Cl. CPC A61B 7/04; A61B 2560/0443; A61B 2562/0204

(57) ABSTRACT

A modular stethoscope includes a first module including a chestpiece and a first tubing disposed in fluid communication with and connected to the chestpiece. The chestpiece is configured to transmit acoustic waves through the first tubing. The modular stethoscope further includes a second module detachably connected to the first module. The second module includes a second tubing and a headset disposed in fluid communication with the second tubing. The modular stethoscope further includes a tube connector fluidly disposed between the first tubing of the first module and the second tubing of the second module. The tube connector is detachably connected to the first tubing of the first module. The tube connector includes a first part, a second part, and a quick coupling member configured to detachably and scalably connect the first part to the second part to acoustically couple the first tubing to the second tubing.

### TECHNICAL FIELD

The present disclosure relates generally to a stethoscope, and in particular, to a modular stethoscope and a method of use thereof.

### BACKGROUND

Stethoscopes are medical devices used for auscultation, primarily in a clinical environment, for listening to internal sounds of a living subject. Due to the nature of the procedure and the environment, stethoscopes may get contaminated with microbes during use. Consequently, the stethoscope may inadvertently become a vector for transmission of infection and microbes between patients and/or between patients and healthcare professionals, and potentially transfer infectious diseases between the patients.

### SUMMARY

In a first aspect, the present disclosure provides a modular stethoscope. The modular stethoscope includes a first module including a chestpiece. The first module further includes a first tubing disposed in fluid communication with and connected to the chestpiece. The chestpiece is configured to transmit acoustic waves through the first tubing. The modular stethoscope further includes a second module detachably connected to the first module. The second module includes a second tubing and a headset disposed in fluid communication with the second tubing. The modular stethoscope further includes a tube connector fluidly disposed between the first tubing of the first module and the second tubing of the second module. The tube connector is detachably connected to the first tubing of the first module. The tube connector includes a first part including a barbed portion configured to be at least partially received within the first tubing to connect the first part to the first tubing. The first part defines a first channel extending therethrough, such that the first channel is configured to be disposed in fluid communication with the first tubing. The tube connector further includes a second part separate from the first part. The second part defines a second channel extending therethrough, such that the second channel is configured to be disposed in fluid communication with the second tubing. The tube connector further includes a quick coupling member configured to detachably and sealably connect the first part to the second part, such that the first channel fluidly communicates with the second channel to acoustically couple the first tubing to the second tubing.

In a second aspect, the present disclosure provides a method of using the modular stethoscope. The method includes connecting the first tubing to the first part of the tube connector. The method further includes fluidly communicating the second tubing to the second part of the tube connector. The method further includes detachably connecting the first part to the second part by the quick coupling member.

In a third aspect, the present disclosure provides a modular stethoscope. The modular stethoscope includes a first module including a chestpiece and a tubing disposed in fluid communication with and connected to the chestpiece. The chestpiece is configured to transmit acoustic waves through the tubing. The first module further includes an electronic module detachably connected to and disposed in fluid communication with the tubing. The electronic module is configured to receive the acoustic waves from the tubing and electronically process the acoustic waves to generate processed acoustic waves. The modular stethoscope further includes a second module including a headset disposed in wireless communication with the electronic module. The headset is configured to receive the processed acoustic waves from the electronic module.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 illustrates a schematic perspective view of a modular stethoscope according to an embodiment of the present disclosure:

FIGS. 2A and 2B illustrate schematic perspective views of a tube connector of the modular stethoscope according to an embodiment of the present disclosure:

FIG. 3A illustrates a schematic sectional side view of a first tubing of the modular stethoscope according to an embodiment of the present disclosure:

FIG. 3B illustrates a schematic sectional side view of a second tubing of the modular stethoscope according to an embodiment of the present disclosure:

FIGS. 4A and 4B illustrate schematic perspective views of a first part of the tube connector according to an embodiment of the present disclosure:

FIGS. 5A and 5B illustrate schematic perspective views of a second part of the tube connector according to an embodiment of the present disclosure:

FIG. 6A illustrates a schematic sectional side view of the first part of the tube connector according to an embodiment of the present disclosure:

FIG. 6B illustrates a schematic sectional side view of the second part of the tube connector according to an embodiment of the present disclosure:

FIG. 7 illustrates a schematic perspective view of a modular stethoscope according to an embodiment of the present disclosure:

FIG. 8 illustrates a block diagram depicting a transmission of acoustic waves in the modular stethoscopes of FIGS. 1 and 7;

FIG. 9 illustrates a schematic perspective view of a modular stethoscope according to an embodiment of the present disclosure:

FIG. 10 illustrates a schematic top view of a modular stethoscope according to an embodiment of the present disclosure;

FIGS. 11A and 11B illustrate schematic perspective views of a first part of a third tube connector according to an embodiment of the present disclosure:

FIGS. 11C and 11D illustrate schematic perspective views of a second part of a first tube connector according to an embodiment of the present disclosure:

FIGS. 12A and 12B illustrate schematic perspective views of an electronic module of the modular stethoscope of FIG. 10 according to an embodiment of the present disclosure;

FIG. 13 illustrates a block diagram depicting components of the electronic module according to an embodiment of the present disclosure:

FIG. 14 illustrates a schematic top view of a modular stethoscope according to an embodiment of the present disclosure;

FIG. 15 illustrates a schematic top view of a modular stethoscope according to an embodiment of the present disclosure:

FIG. 16 illustrates a schematic top view of a modular stethoscope according to an embodiment of the present disclosure;

FIG. 17 illustrates a block diagram depicting a transmission of the acoustic waves in the modular stethoscope of FIG. 16 according to an embodiment of the present disclosure:

FIG. 18 illustrates a schematic perspective view of a modular stethoscope according to an embodiment of the present disclosure;

FIG. 19 illustrates a block diagram depicting a transmission of the acoustic waves in the modular stethoscope of FIG. 18 according to an embodiment of the present disclosure:

FIG. 20 illustrates a schematic perspective view of a modular stethoscope according to an embodiment of the present disclosure;

FIG. 21 illustrates a block diagram depicting components of an electric chestpiece of the modular stethoscope of FIG. 20 according to an embodiment of the present disclosure:

FIG. 22 illustrates a block diagram depicting a transmission of the acoustic waves in the modular stethoscope of FIG. 20 according to an embodiment of the present disclosure;

FIG. 23 illustrates a flow chart depicting a method of using the modular stethoscope according to an embodiment of the present disclosure; and

FIG. 24 illustrates a block diagram of a modular stethoscope according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, layers, components, or elements may be described as being adjacent one another. Layers, components, or elements can be adjacent one another by being in direct contact, by being connected through one or more other components, or by being held next to one another or attached to one another. Layers, components, or elements that are in direct contact may be described as being immediately adjacent or directly adjacent.

By using words of orientation such as “on”, “uppermost” it is referred to the relative position of one or more particle with respect to a horizontal support layer.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “similar” to a second material, at least 90 weight % of the first and second materials are identical and any variation between the first and second materials comprises less than about 10 weight % of each of the first and second materials.

The present disclosure relates to a modular stethoscope. The modular stethoscope may be used for auscultation by a user. In some cases, the user may be a health worker or a medical personnel.

The modular stethoscope includes a first module including a chestpiece. The first module further includes a first tubing disposed in fluid communication with and connected to the chestpiece. The chestpiece is configured to transmit acoustic waves through the first tubing. The modular stethoscope further includes a second module detachably connected to the first module. The second module includes a second tubing and a headset disposed in fluid communication with the second tubing. The modular stethoscope further includes a tube connector fluidly disposed between the first tubing of the first module and the second tubing of the second module. The tube connector is detachably connected to the first tubing of the first module. The tube connector includes a first part including a barbed portion configured to be at least partially received within the first tubing to connect the first part to the first tubing. The first part defines a first channel extending therethrough, such that the first channel is configured to be disposed in fluid communication with the first tubing. The tube connector further includes a second part separate from the first part. The second part defines a second channel extending therethrough, such that the second channel is configured to be disposed in fluid communication with the second tubing. The tube connector further includes a quick coupling member configured to detachably and scalably connect the first part to the second part, such that the first channel fluidly communicates with the second channel to acoustically couple the first tubing to the second tubing.

Conventional stethoscopes may not allow changing of different parts that make up the conventional stethoscopes. Therefore, in some cases, the conventional stethoscopes may transfer microbes between patients, and potentially transfer infectious diseases between the patients. Furthermore, conventional stethoscopes that are disposable (i.e., disposable conventional stethoscopes) typically provide inferior sound quality, fit, and noise isolation than the non-modular conventional stethoscopes. Moreover, the conventional stethoscopes may be difficult to use while wearing personal protective equipment (e.g., hood, gown, mask, googles, face shield, gloves, etc.).

The modular stethoscope of the present disclosure may allow a user to use the second module including the headset with the first module including the chestpiece. The first module may include a variety of configurations. For example, a mechanical chestpiece and an electrical/digital chestpiece may be used with the second module. Advantageously, a replacement and/or backup chestpiece may be used in case of failure of the chestpiece. Further, the tube connector including the quick coupling member may allow the user to quickly attach and detach the first module with the second module. Further, the tube connector including the quick coupling member may allow the user to quickly attach and detach the first module with the second module using one hand. Moreover, the tube connector including the quick coupling member may allow toolless coupling and decoupling of the first module to and from the second module, and therefore may facilitate attachment and detachment of the first module to and from the second module using one hand. In some cases, the modular stethoscope may include an electronic module in order to process the acoustic waves transmitted by the chestpiece. Specifically, the electronic module may generate processed acoustic waves that may improve at least one characteristic (e.g., volume, noise level, etc.) of the acoustic waves.

The modular stethoscope of the present disclosure may be safely used with an infectious patient. The user may quickly attach the first module that is disposable/semi-disposable when using the modular stethoscope with the infectious patient. Further, the user may dispose of the first module or store it for future use with the infectious patient. In some cases, the user may send the first module for hospital reprocessing. The second module of the modular stethoscope may remain effectively clean, and may be used with a non-infectious patient by attaching a different first module with the help of the tube connector with the second module.

In other words, the first module may directly contact the patient. Therefore, the first module may be treated as a hazardous element and disposed after single-use. Furthermore, the second module may not directly contact the patient. Therefore, the second module may be cleaned/disinfected with less rigorous means and reused. Moreover, the second module of the modular stethoscope may be worn by the user under the personal protective equipment, and therefore may be accessible by the user. In some cases, the personal protective equipment may include a port/hole, and only a small portion of the second module may be exposed outside the personal protective equipment. Consequently, the modular stethoscope may protect both the patient and the user from inadvertent exposure to microbial contamination from the medical device.

Therefore, the modular stethoscope of the present disclosure may provide accessibility when used with the personal protective equipment, protection from cross-contamination in infectious environments, and freedom to choose different types of first modules including different chestpieces, as per desired application attributes. The modular stethoscope may further allow the user to quickly attach and detach the first module with the second module using one hand, while providing a secure and reliable connection between the first and second modules.

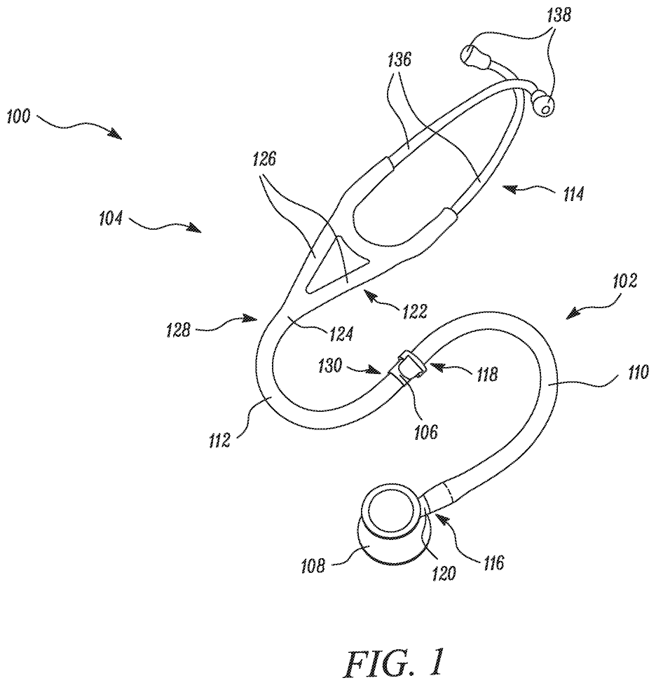

Referring now to the figures, FIG. 1 illustrates a modular stethoscope 100 according to an embodiment of the present disclosure. The modular stethoscope 100 includes a first module 102, a second module 104, and a tube connector 106. Hereinafter, the tube connector 106 may be interchangeably referred to as “the first tube connector 106”.

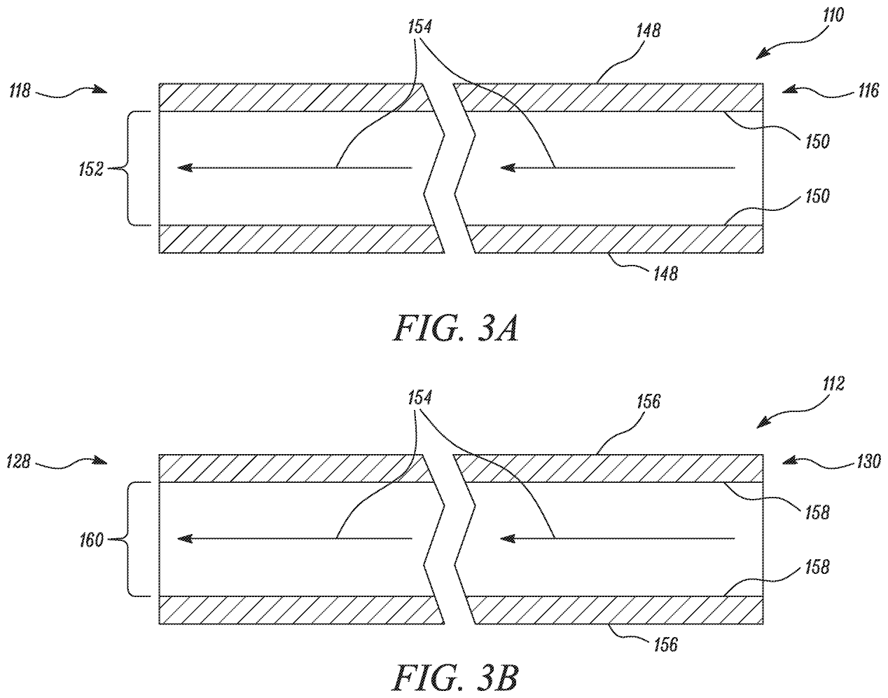

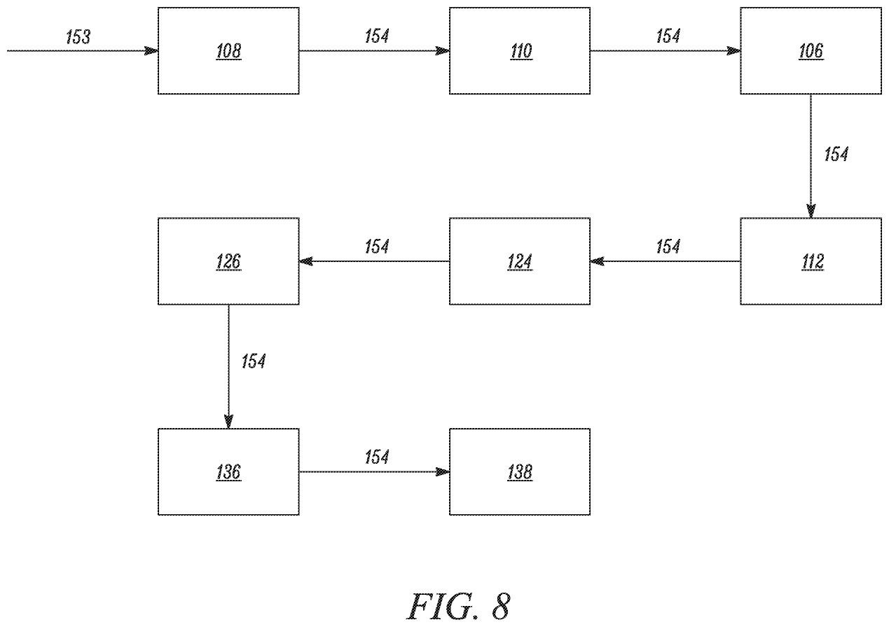

The first module 102 includes a chestpiece 108. The chestpiece 108 may be placed on a region of a body of a patient (e.g., chest, back, abdomen, etc.) requiring auscultation by a user. In some cases, the user may be a health worker. The first module 102 further includes a first tubing 110 disposed in fluid communication with and connected to the chestpiece 108. The chestpiece 108 may be configured to receive a sound 153 (shown in FIG. 8). The chestpiece 108 is configured to transmit acoustic waves 154 (shown in FIG. 3A) through the first tubing 110. Specifically, the chestpiece 108 may be configured to receive the sound 153 and transmit the sound 153 as the acoustic waves 154.

As shown in FIG. 1, in some embodiments, the first tubing 110 includes a first proximal end 116 connected to the chestpiece 108. In some embodiments, the first tubing 110 further includes a first distal end 118 opposite to the first proximal end 116 and distal to the chestpiece 108. In some embodiments, the chestpiece 108 includes a projection 120 that is at least partially received within the first tubing 110 to detachably connect the chestpiece 108 to the first tubing 110. Specifically, in some embodiments, the projection 120 may be at least partially received within the first tubing 110 at the first proximal end 116 of the first tubing 110. The projection 120 may include a suitable structure that may be at least partially received within the first tubing 110 to detachably and fluidly connect the chestpiece 108 to the first tubing 110. In some embodiments, the projection 120 may include one or more barbs. An end of the projection 120 that is received within the first tubing 110 is shown by dashed lines in FIG. 1.

The second module 104 is detachably connected to the first module 102. The second module 104 includes a second tubing 112 and a headset 114 disposed in fluid communication with the second tubing 112. In some embodiments, the second tubing 112 may be an extension tubing. In other words, the second tubing 112 may permit lengthening of the modular stethoscope 100 to create additional distance between the patient and the user. Furthermore, the second tubing 112 may be extended from beneath personal protective equipment worn by the user. With additional COVID personal protective equipment, length of the conventional stethoscopes may substantially restrict movement of the user, thus, making it more difficult to auscultate effectively without contamination risk. The second tubing 112 of the modular stethoscope 100 may allow the user to be at a greater distance from the patient during auscultation by the user. Therefore, the modular stethoscope 100 may allow the user to auscultate effectively without contamination risk, thereby further protecting both the patient and the user.

Acoustical testing has shown that lengthening the tubing for use with personal protective equipment can have a slight shift in the resonance frequency. Testing has shown that if the total tubing length is increased from 20.2 inches to 40.4 inches there is a frequency shift in the resonance peak from 82 Hz to 68 Hz due to the added tubing length. This accoustal effect is inconsequential to the user and would likely not be noticed. Extended length tubing for use with personnel protective equipment can be useful with the modular systems shown in the various figures herein, or simply with a conventional stethoscope having a chest piece and tubing connecting the chest piece to a headset (binaurals).

As shown in FIG. 1, in some embodiments, the headset 114 includes a yoke 122 including an inlet tube 124 disposed proximal to and in fluid communication with the second tubing 112. In the illustrated embodiment of FIG. 1, the yoke 122 is integrally formed with the second tubing 112, such that the inlet tube 124 is integral with the second tubing 112. However, in some other embodiments, the yoke 122 may be formed separately from the second tubing 112, such that the inlet tube 124 of the yoke 122 is separate from the second tubing 112. In other words, in some other embodiments, the inlet tube 124 may not be integral with the second tubing 112.

In some embodiments, the yoke 122 further includes a pair of outlet tubes 126 disposed in fluid communication with the inlet tube 124. The pair of outlet tubes 126 is disposed distal to the second tubing 112. The yoke 122 including the inlet tube 124 and the pair of outlet tubes 126 may have a substantially Y-shaped configuration. In the illustrated embodiment of FIG. 1, the headset 114 further includes a pair of car tubes 136, and a pair of earpieces 138. Each of the pair of car tubes 136 is connected to a corresponding outlet tube 126 of the pair of outlet tubes 126 of the yoke 122. Further, each of the pair of earpieces 138 is connected to a corresponding car tube 136 of the pair of car tubes 136.

As shown in FIG. 1, in some embodiments, the second tubing 112 includes a second proximal end 128 connected to the headset 114. In some embodiments, the second tubing 112 further includes a second distal end 130 opposite to the second proximal end 128 and distal to the headset 114. In some embodiments, a length of the first tubing 110 is greater than or equal to a length of the second tubing 112. In the illustrated embodiment of FIG. 1, the length of the first tubing 110 is greater than the length of the second tubing 112. However, in some other embodiments, the length of the first tubing 110 may be less than the length of the second tubing 112. In some embodiments, a ratio of the length of the first tubing 110 to the length of the second tubing 112 is at least 3.

The tube connector 106 is fluidly disposed between the first tubing 110 of the first module 102 and the second tubing 112 of the second module 104. The tube connector 106 is detachably connected to the first tubing 110 of the first module 102. Specifically, in the illustrated embodiment of FIG. 1, the tube connector 106 is detachably connected to the first tubing 110 proximal to the first distal end 118 and distal to the chestpiece 108. Further, the tube connector 106 is detachably connected to the second tubing 112 proximal to the second distal end 130 and distal to the headset 114.

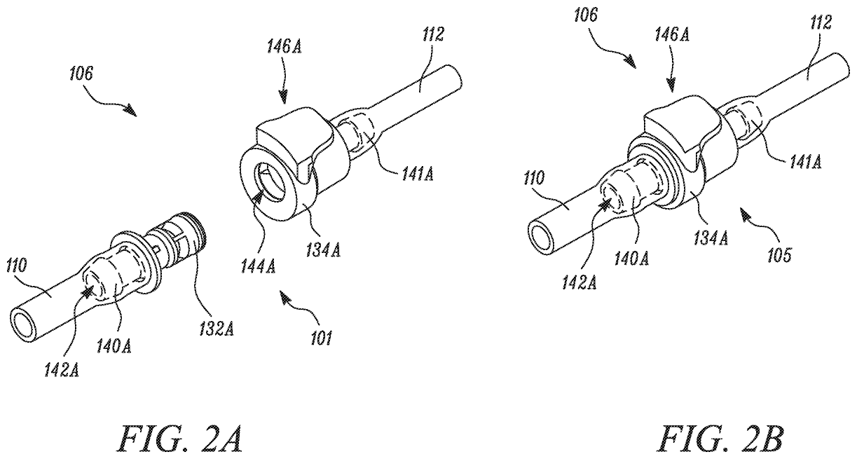

FIGS. 2A and 2B illustrate the tube connector 106 in a disconnected state 101 and a connected state 105, respectively. Referring to FIGS. 1, 2A, and 2B, the tube connector 106 includes a first part 132A, a second part 134A separate from the first part 132A, and a quick coupling member 146A. The quick coupling member 146A is configured to detachably and sealably connect the first part 132A to the second part 134A.

The first part 132A includes a barbed portion 140A configured to be at least partially received within the first tubing 110 to connect the first part 132A to the first tubing 110. In some embodiments, the barbed portion 140A of the first part 132A includes one or more barbs. In the illustrated embodiment of FIGS. 2A and 2B, the barbed portion 140A of the first part 132A includes one barb. In some other embodiments, the barbed portion 140A may include more than two, more than three, or more than four barbs, as per desired application attributes. The first part 132A defines a first channel 142A extending therethrough, such that the first channel 142A is configured to be disposed in fluid communication with the first tubing 110.

The second part 134A defines a second channel 144A extending therethrough, such that the second channel 144A is configured to be disposed in fluid communication with the second tubing 112. In some embodiments, the second part 134A may include a barbed portion or a threaded portion. In the illustrated embodiment of FIGS. 2A and 2B, the second part 134A includes the barbed portion to connect the second part 134A to a tubing. In some other embodiments, the second part 134A may include the threaded portion to detachably and threadably connect the second part 134A to additional components that may be included in the modular stethoscope 100 (shown in FIG. 1).

In the illustrated embodiment of FIGS. 2A and 2B, the second part 134A includes a barbed portion 141A configured to be at least partially received within the second tubing 112 to connect the second part 134A to the second tubing 112. In some embodiments, the barbed portion 141A of the second part 134A includes one or more barbs. In the illustrated embodiment of FIGS. 2A and 2B, the barbed portion 141A of the second part 134A includes one barb. However, in some other embodiments, the barbed portion 141A may have more than two, more than three, or more than four barbs, as per desired application attributes.

The quick coupling member 146A is configured to detachably and scalably connect the first part 132A to the second part 134A, such that the first channel 142A fluidly communicates with the second channel 144A to acoustically couple the first tubing 110 to the second tubing 112.

FIGS. 3A and 3B illustrate schematic sectional side views of the first tubing 110 and the second tubing 112, respectively. Referring to FIG. 3A, in some embodiments, the first tubing 110 further includes a first outer surface 148 and a first inner surface 150. Each of the first outer surface 148 and the first inner surface 150 extends between the first proximal end 116 and the first distal end 118. The first inner surface 150 defines a first inner volume 152 configured to transport the acoustic waves 154. The first inner volume 152 of the first inner surface 150 may vary as per desired application attributes. In some embodiments, the barbed portion 140A (shown in FIGS. 2A and 2B) of the first part 132A of the tube connector 106A is configured to be at least partially received within the first inner surface 150 of the first tubing 110 at the first distal end 118.

Referring to FIG. 3B, in some embodiments, the second tubing 112 further includes a second outer surface 156 and a second inner surface 158. Each of the second outer surface 156 and the second inner surface 158 extends between the second proximal end 128 and the second distal end 130. The second inner surface 158 defines a second inner volume 160 configured to transport the acoustic waves 154. The second inner volume 160 of the second inner surface 158 may vary as per desired application attributes. In some embodiments, the barbed portion 141A (shown in FIGS. 2A and 2B) of the second part 134A of the tube connector 106 may be configured to be at least partially received within the second inner surface 158 of the second tubing 112 at the second distal end 130.

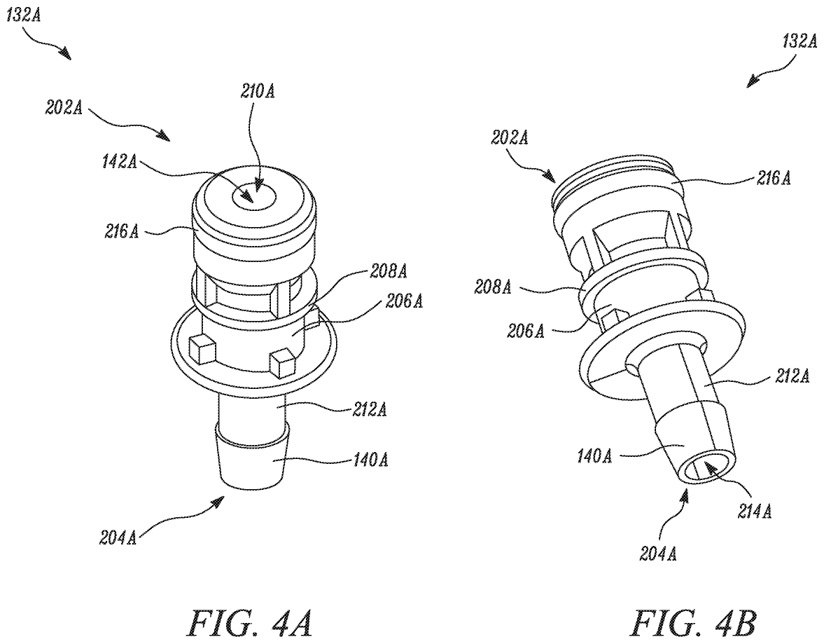

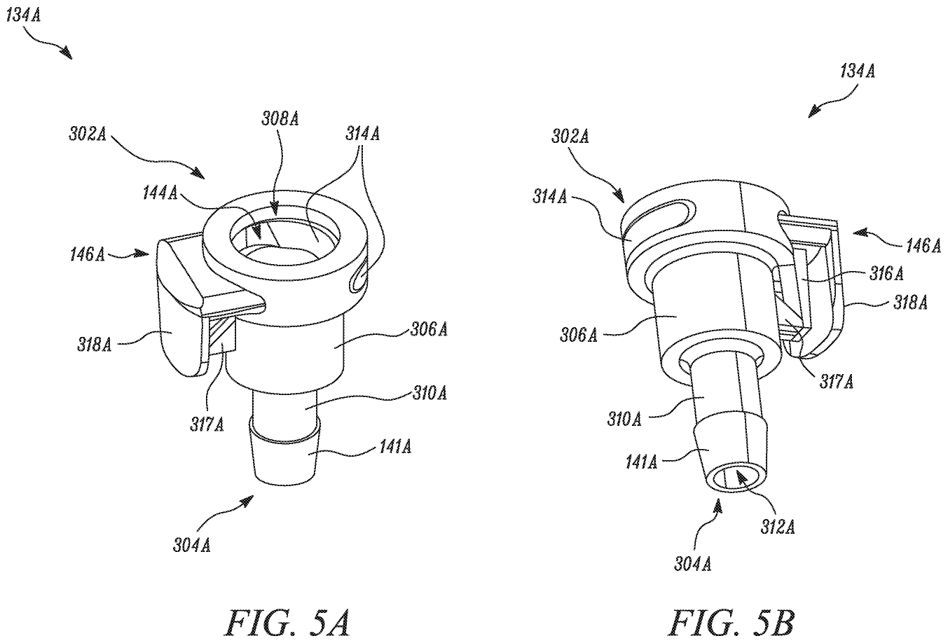

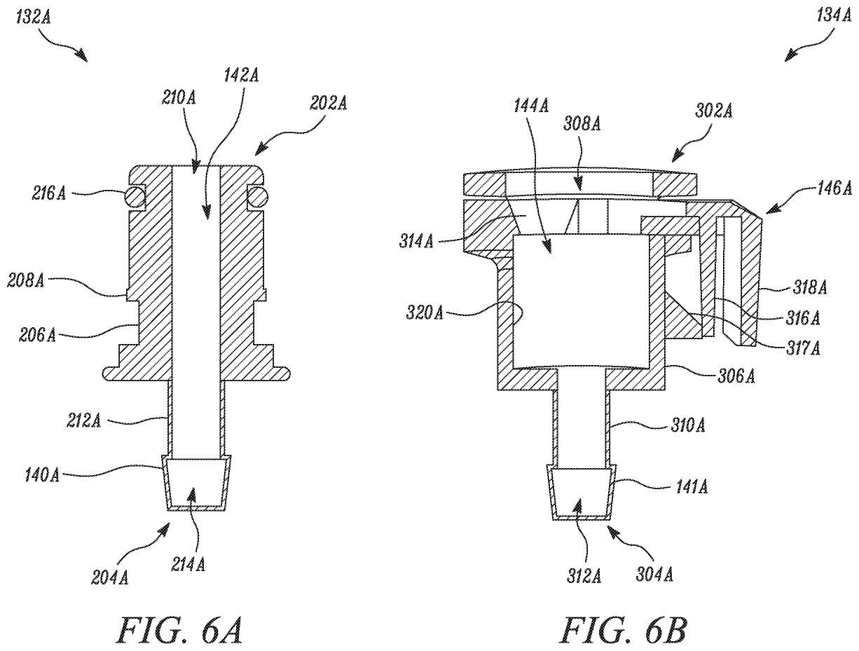

FIGS. 4A and 4B illustrate the first part 132A of the tube connector 106 (shown in FIG. 1) according to an embodiment of the present disclosure. Further, FIGS. 5A and 5B illustrate the second part 134A of the tube connector 106 according to an embodiment of the present disclosure. Moreover, FIGS. 6A and 6B illustrate sectional side views of the first part 132A and the second part 134A of the tube connector 106, respectively, according to an embodiment of the present disclosure.

Referring to FIGS. 1, 4A, 4B, and 6A, as discussed above, the first part 132A includes the barbed portion 140A configured to be at least partially received within the first tubing 110 (shown in FIGS. 2A and 2B) to connect the first part 132A to the first tubing 110. Further, the first part 132A defines the first channel 142A extending therethrough, such that the first channel 142A is configured to be disposed in fluid communication with the first tubing 110.

In some embodiments, the first part 132A of the tube connector 106 further includes a first end 202A and a second end 204A opposing the first end 202A, such that the first channel 142A extends from the first end 202A to the second end 204A. In some embodiments, the first part 132A of the tube connector 106 further includes a first main body 206A including the first end 202A and an annular projection 208A. The first main body 206A defines a first through opening 210A at the first end 202A fluidly communicating with the first channel 142A.

In some embodiments, the first part 132A of the tube connector 106 further includes a first connecting body 212A extending from the first main body 206A and including the second end 204A and the barbed portion 140A. The first connecting body 212A defines a second through opening 214A (shown in FIG. 4B) at the second end 204A fluidly communicating with the first channel 142A.

In some embodiments, the first part 132A of the tube connector 106 further includes a scaling member 216A mounted on the first main body 206A proximal to the first end 202A. The scaling member 216A may be made of a suitable material, as per desired application attributes. In some embodiments, the scaling member 216A may be made of a flexible material, such as a polymeric material, in particular a thermosetting material, a thermoplastic material, an elastomer, a resin, and combinations thereof. The scaling member 216A may provide a substantially air-tight compression seal when the second part 134A is detachably connected to the first part 132A (shown in FIG. 2B). The air-tight compression seal may provide improved quality of the acoustic waves 154 (shown in FIGS. 3A and 3B) transmitted through the tube connector 106. In other words, the sealing member 216A may ensure that the acoustic waves 154 are transmitted through the tube connector 106 without leakage or obstruction.

Now referring to FIGS. 5A, 5B, and 6B, in some embodiments, the second part 134A of the tube connector 106 (shown in FIGS. 2A and 2B) includes a first end 302A and a second end 304A opposing the first end 302A, such that the second channel 144A extends from the first end 302A to the second end 304A. In some embodiments, the second part 134A of the tube connector 106 further includes a second main body 306A including the first end 302A and defining a first through opening 308A at the first end 302A fluidly communicating with the second channel 144A.

As shown in FIGS. 5A, 5B, and 6B, in some embodiments, the second part 134A of the tube connector 106 further includes a second connecting body 310A extending from the second main body 306A and including the second end 304A. In some embodiments, the second connecting body 310A includes a barbed portion or a threaded portion. In the illustrated embodiment of FIGS. 5A, 5B, and 6B, the second connecting body 310A includes the barbed portion 141A.

The second connecting body 310A defines a second through opening 312A (shown in FIG. 5B) at the second end 304A fluidly communicating with the second channel 144A. The second main body 306A and the second connecting body 310A together define the second channel 144A extending through the second main body 306A and the second connecting body 310A.

In some embodiments, the second part 134A of the tube connector 106 further includes the quick coupling member 146A movably mounted on the second main body 306A. The quick coupling member 146A includes a collar 314A movable between a connected position and a disconnected position. The quick coupling member 146A further includes a biasing portion 316A (shown in FIG. 5B) configured to bias the collar 314A to the connected position. In some embodiments, the biasing portion 316A may be a resilient biasing member configured to bias the collar 314A to the connected position. The quick coupling member 146A further includes a tab 318A coupled to the collar 314A and configured to move the collar 314A from the connected position to the disconnected position against a biasing of the biasing portion 316A. Specifically, the tab 318A may provide a surface for the user to move the collar 314A from the connected position to the disconnected position.

In some embodiments, the second main body 306A further includes a stop member 317A configured to engage the biasing portion 316A. Specifically, upon applying a force on the tab 318A opposite to and against the biasing of the biasing portion 316A, the biasing portion 316A may be resiliently displaced and may engage the stop member 317A, such that the collar 314A resiliently moves from the connected position to the disconnected position. Further, upon removal of the force applied on the tab 318A, the biasing portion 316A may bias the collar 314A from the disconnected position to the connected position.

Referring to FIGS. 1, 6A, and 6B, in some embodiments, the first main body 206A is at least partially received within the second main body 306A of the second part 134A through the first through opening 308A. In the connected position, the collar 314A of the quick coupling member 146A engages with the annular projection 208A of the first main body 206A to secure the first main body 206A to the second main body 306A. Further, the sealing member 216A engages an inner surface 320A (shown in FIG. 6B) of the second main body 306A upon detachably connecting the first main body 206A to the second main body 306A. In the disconnected position, the collar 314A of the quick coupling member 146A allows the first main body 206A to be removed from the second main body 306A. As discussed above, the collar 314A of the quick coupling member 146A may move from the connected position to the disconnected position upon applying the force on the tab 318A opposite to and against the biasing of the biasing portion 316A.

Referring to FIGS. 2A and 2B, in some embodiments, the first part 132A and the second part 134A of the tube connector 106 may be interchangeably used. For example, the barbed portion 140A of the first part 132A may be configured to be at least partially received within the second tubing 112 to connect the first part 132A to the second tubing 112. Further, the barbed portion 141A of the second part 134A may be configured to be at least partially received within the first tubing 110 to connect the second part 134A to the first tubing 110.

The first part 132A and the second part 134A may be part of other tube connectors (in addition to the tube connector 106) used in different configurations of modular stethoscopes of the present disclosure. The reference numerals associated with the first and second parts 132A, 134A may be reused or counterpart reference numerals may be used when describing the other tube connectors.

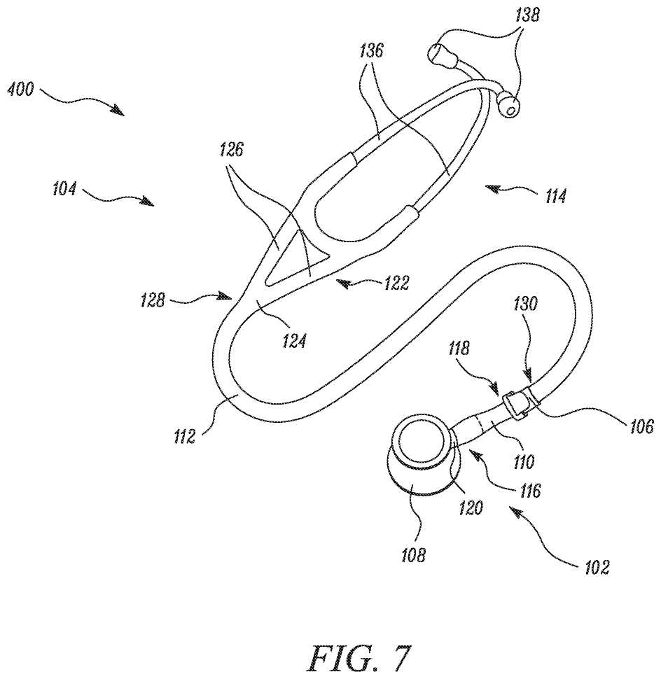

FIG. 7 illustrates a modular stethoscope 400 according to another embodiment of the present disclosure. The modular stethoscope 400 is substantially similar to the modular stethoscope 100 of FIG. 1. However, the relative lengths of the first tubing 110 and the second tubing 112 of the modular stethoscope 400 may be different from that of the modular stethoscope 100. In the illustrated embodiment of FIG. 7, the chestpiece 108 is a mechanical chestpiece. The mechanical chestpiece may include a diaphragm configured to produce the acoustic waves 154 (shown in FIGS. 3A and 3B) from the sound 153 (shown in FIG. 8) during auscultation by the user. Further, in the illustrated embodiment of FIG. 7, the length of the first tubing 110 is less than the length of the second tubing 112. Therefore, in the illustrated embodiment of FIG. 7, the ratio of the length of the first tubing 110 to the length of the second tubing 112 is less than 1. Specifically, the ratio of the length of the first tubing 110 to the length of the second tubing 112 is less than about 0.25. In other words, the length of the second tubing 112 is at least four times the length of the first tubing 110.

FIG. 8 illustrates a block diagram depicting a transmission of the acoustic waves 154 in the modular stethoscopes 100, 400 of FIGS. 1 and 7, respectively, according to an embodiment of the present disclosure. Referring to FIGS. 1, 7, and 8, the chestpiece 108 receives the sound 153. The sound 153 may be from the region of the body of the patient undergoing auscultation by the user. In some embodiments, the chestpiece 108 transmits the sound 153 as the acoustic waves 154. As discussed above, the chestpiece 108 is configured to transmit the acoustic waves 154 through the first tubing 110.

Since the tube connector 106 is fluidly disposed between the first tubing 110 of the first module 102 and the second tubing 112 of the second module 104, the acoustic waves 154 are further transmitted through the tube connector 106 to the second tubing 112. In the illustrated embodiments of FIGS. 1 and 7, the headset 114 includes the yoke 122 including the inlet tube 124 disposed proximal to and in fluid communication with the second tubing 112. The yoke 122 further includes the pair of outlet tubes 126 disposed in fluid communication with the inlet tube 124. Moreover, the headset 114 further includes the pair of car tubes 136. Each of the pair of car tubes 136 is connected to the corresponding outlet tube 126 of the pair of outlet tubes 126. Therefore, the acoustic waves 154 are further transmitted through the inlet tube 124 of the yoke 122 to the pair of outlet tubes 126. The acoustic waves 154 are further transmitted through the pair of car tubes 136, and finally to the pair of earpieces 138. The user may receive the sound 153 as the acoustic waves 154 through the pair of earpieces 138.

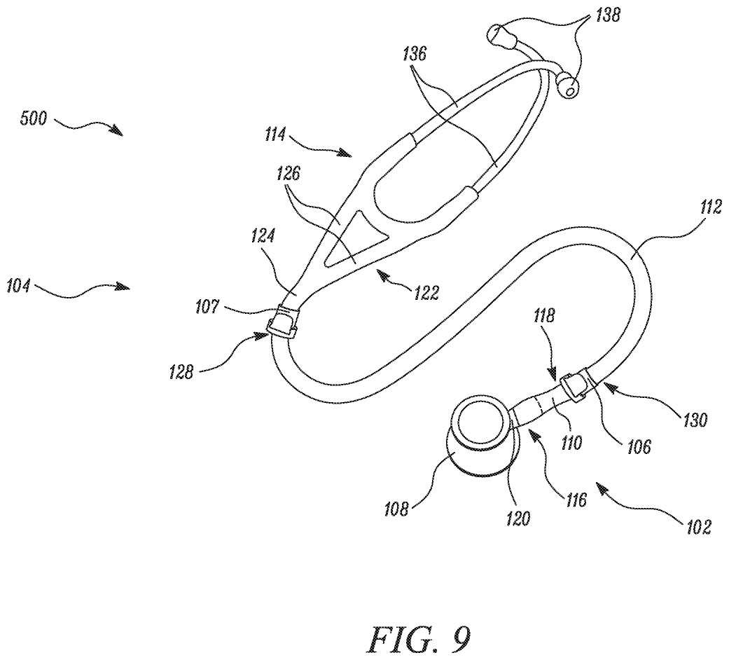

FIG. 9 illustrates a modular stethoscope 500 according to another embodiment of the present disclosure. The modular stethoscope 500 has one or more structures and functions that are substantially similar to those of the modular stethoscope 100 of FIG. 1. However, the modular stethoscope 500 has a different configuration from the modular stethoscope 100 with regards to a connection of the second tubing 112 with the inlet tube 124 of the yoke 122. Specifically, in the illustrated embodiment of FIG. 9, the yoke 122 is formed separately from the second tubing 112, such that the inlet tube 124 is separate from the second tubing 112. In other words, the inlet tube 124 is not integral with the second tubing 112.

In the illustrated embodiment of FIG. 9, the tube connector 106 is fluidly disposed between the first tubing 110 of the first module 102 and the second tubing 112 of the second module 104. In other words, the first tube connector 106 is fluidly disposed between the first tubing 110 of the first module 102 and the second tubing 112 of the second module 104. The first tube connector 106 acoustically couples the first tubing 110 and the second tubing 112.

The modular stethoscope 500 further includes a second tube connector 107 fluidly disposed between the second tubing 112 and the headset 114. The second tube connector 107 is substantially similar to the first tube connector 106 shown in FIGS. 2A-6B. Therefore, the same reference characters that are used to describe the first tube connector 106 are used to describe the second tube connector 107. Specifically, the second channel 144A of the second part 134A of the second tube connector 107 is configured to be disposed in fluid communication with the inlet tube 124, instead of being disposed in fluid communication with the second tubing 112.

Referring to FIGS. 4A-6B and 9, the second tube connector 107 includes the first part 132A, the second part 134A separate from the first part 132A, and the quick coupling member 146A. The quick coupling member 146A is configured to detachably and scalably connect the first part 132A of the second tube connector 107 to the second part 134A of the second tube connector 107.

The first part 132A of the second tube connector 107 includes the barbed portion 140A configured to be at least partially received within the second tubing 112 to connect the first part 132A to the second tubing 112. The first part 132A defines the first channel 142A extending therethrough, such that the first channel 142A is configured to be disposed in fluid communication with the second tubing 112.

The second part 134A of the second tube connector 107 includes the barbed portion 141A configured to be at least partially received within the inlet tube 124 of the headset 114 to connect the second part 134A to the inlet tube 124. In other words, the second part 134A includes the barbed portion 141A configured to be at least partially received within the inlet tube 124 of the yoke 122 to connect the second part 134A to the inlet tube 124. The second part 134A defines the second channel 144A extending therethrough, such that the second channel 144A is configured to be disposed in fluid communication with the inlet tube 124.

The quick coupling member 146A of the second tube connector 107 is configured to detachably and sealably connect the first part 132A of the second tube connector 107 to the second part 134A of the second tube connector 107, such that the first channel 142A fluidly communicates with the second channel 144A to acoustically couple the second tubing 112 to the inlet tube 124 of the headset 114. Specifically, the quick coupling member 146 is configured to detachably and scalably connect the first part 132A of the second tube connector 107 to the second part 134A of the second tube connector 107, such that the first channel 142A fluidly communicates with the second channel 144A to acoustically couple the second tubing 112 to the inlet tube 124 of the yoke 122. As discussed above, the first part 132A and the second part 134A may be interchangeably used to acoustically couple the second tubing 112 to the inlet tube 124 of the yoke 122.

In some embodiments, a ratio of the length of the second tubing 112 to a length of the inlet tube 124 of the yoke 122 is at least 3. In some embodiments, the ratio of the length of the second tubing 112 to the length of the inlet tube 124 of the yoke 122 is at least 4, is at least 5, is at least 6, or is at least 7. However, in some other embodiments, the length of the second tubing 112 is less than or equal to the length of the inlet tube 124 of the yoke 122.

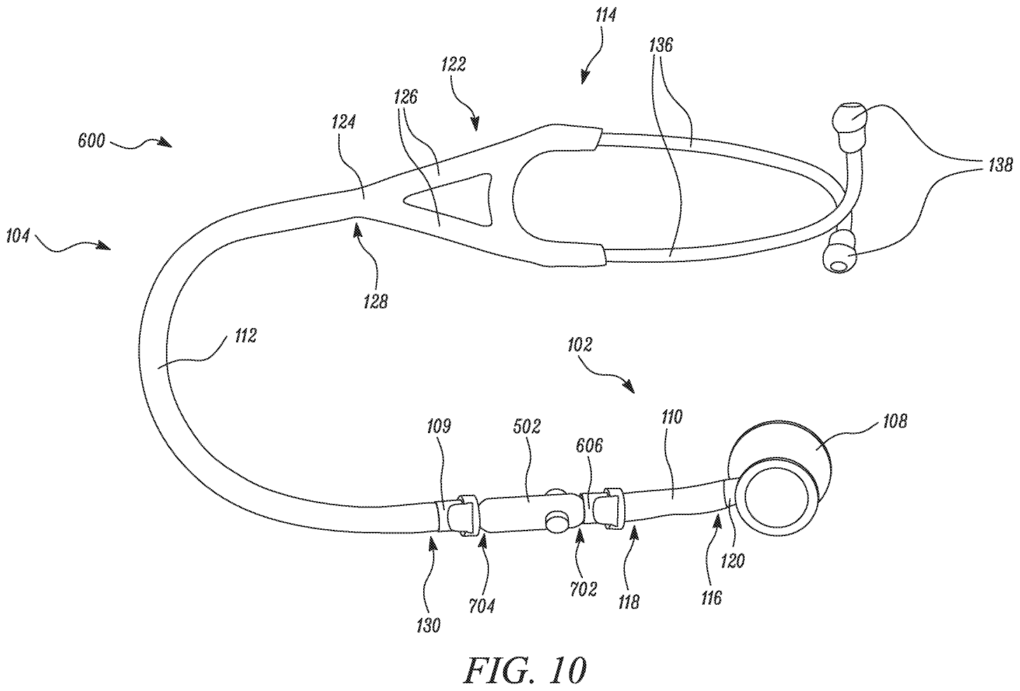

FIG. 10 illustrates a modular stethoscope 600 according to another embodiment of the present disclosure. The modular stethoscope 600 is similar to the modular stethoscope 100 (shown in FIG. 1), with features equivalent to the modular stethoscope 100 designated by like reference numbers. However, in the illustrated embodiment of FIG. 10, the length of the first tubing 110 is less than the length of the second tubing 112. In some embodiments, the length of the second tubing 112 may be at least three times the length of the first tubing 110.

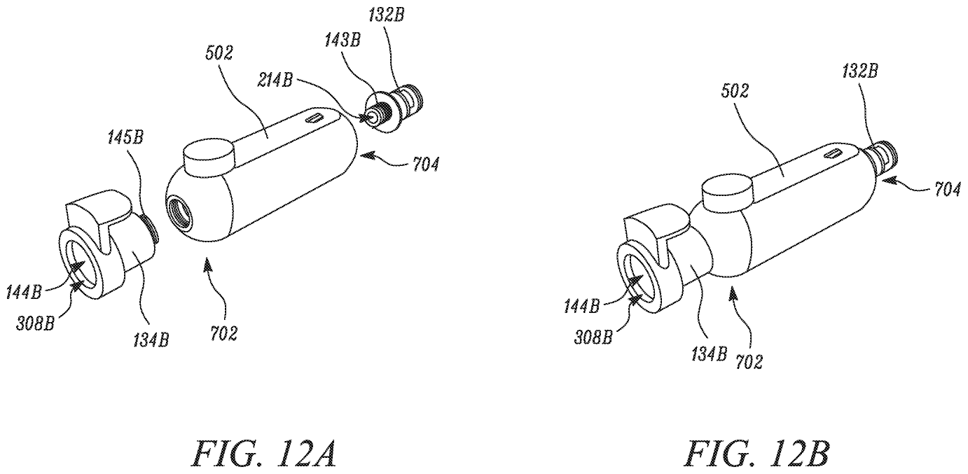

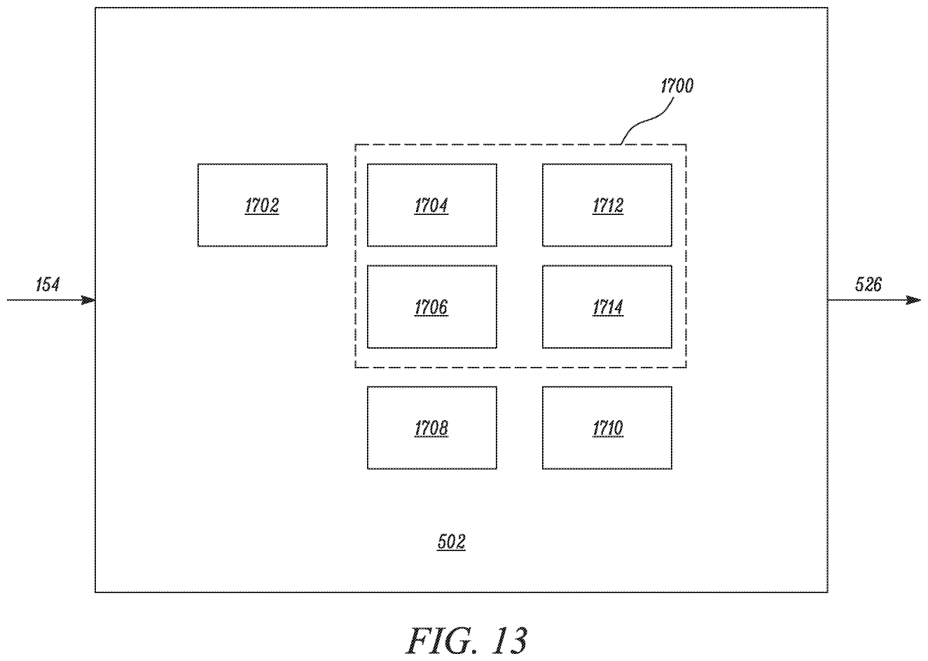

Further, in the illustrated embodiment of FIG. 10, the modular stethoscope 600 further includes an electronic module 502 fluidly disposed between the first module 102 and the second module 104. Referring to FIGS. 10, 12A, and 12B, the electronic module 502 is configured to receive the acoustic waves 154 (shown in FIG. 13) from the first tubing 110 and electronically process the acoustic waves 154 to generate processed acoustic waves 526 (shown in FIG. 13), such that the second tubing 112 receives the processed acoustic waves 526 from the electronic module 502. However, in some other embodiments, the second tubing 112 may be removed, and the processed acoustic waves 526 may be transmitted to a digital headset via wireless communication. This configuration may be ideally suited for use underneath the personal protective equipment designed to protect the user when working in an isolation space. The primary configuration illustrated in FIG. 10, i.e., delivering the processed acoustic waves 526 directly to the cars of the user via the second tubing 112 might put the user at an increased risk of infection, as it may compromise a performance of the personal protective equipment. Specifically, including a port/hole in the personal protective equipment for extending the second tubing 112 outside may compromise the performance of the personal protective equipment.

The electronic module 502 includes a first end 702 proximal to the first module 102, and a second end 704 opposite to the first end 702 and distal to the first module 102. In other words, the second end 704 is proximal to the second module 104.

Referring to FIGS. 10-12B, the modular stethoscope 600 further includes a tube connector 606 fluidly disposed between the first tubing 110 of the first module 102 and the second tubing 112 of the second module 104. The tube connector 606 may be interchangeably referred to as “the first tube connector 606”. In other words, the modular stethoscope 600 further includes the first tube connector 606 fluidly disposed between the first tubing 110 of the first module 102 and the second tubing 112 of the second module 104. The first tube connector 606 is detachably connected to the first tubing 110 of the first module 102.

In the illustrated embodiment of FIG. 10, the first tube connector 606 includes the first part 132A (shown in FIGS. 4A and 4B). The first part 132A includes the barbed portion 140A configured to be at least partially received within the first tubing 110 to connect the first part 132A to the first tubing 110. The first part 132A defines the first channel 142A extending therethrough, such that the first channel 142A is configured to be disposed in fluid communication with the first tubing 110.

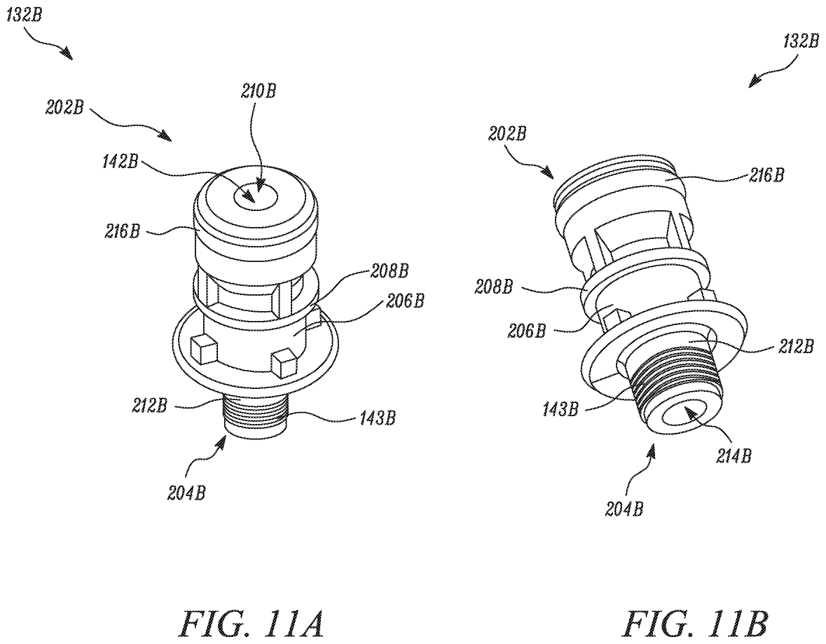

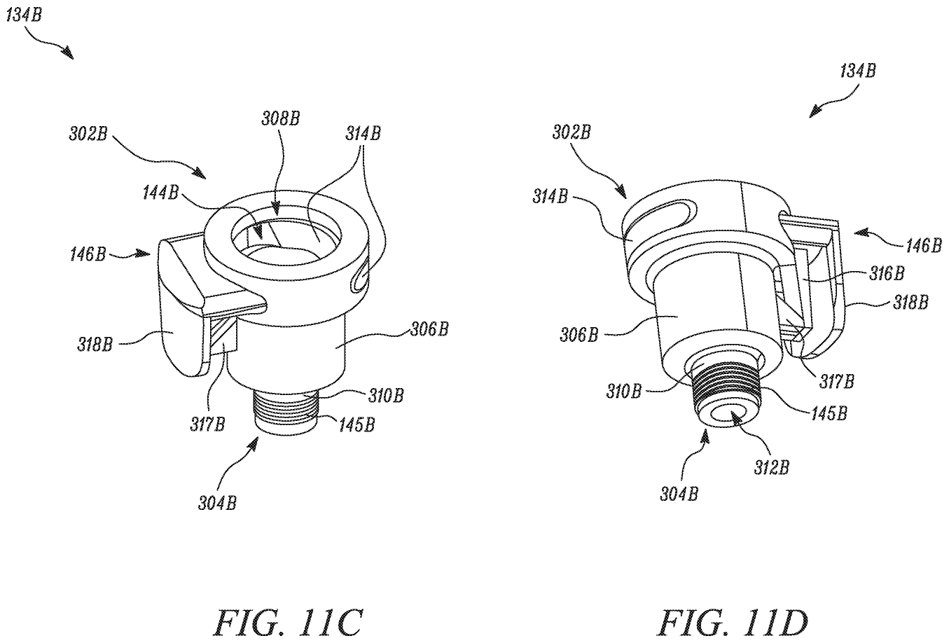

Moreover, in the illustrated embodiment of FIG. 10, the first tube connector 606 further includes a second part 134B (shown in FIGS. 11C and 11D). The second part 134B is substantially similar to the second part 134A (shown in FIGS. 5A and 5B), with features equivalent to those of the second part 134A, shown in FIGS. 5A and 5B, designated by counterpart reference numbers suffixed with “B” instead of “A”. However, the second part 134B includes a threaded portion 145B instead of the barbed portion 141A of the second part 134A (shown in FIGS. 5A and 5B).

Referring to FIGS. 10, 11C, and 11D, the second part 134B of the first tube connector 606 includes the threaded portion 145B configured to be threadably and detachably connected to the first end 702 of the electronic module 502, such that a second channel 144B of the second part 134B of the first tube connector 606 is disposed in fluid communication with the electronic module 502. The electronic module 502 includes corresponding threads (not shown) at the first end 702 for threadably coupling with the threaded portion 145B of the second part 134B of the first tube connector 606.

The first tube connector 606 further includes a quick coupling member 146B (shown in FIGS. 11C and 11D) configured to detachably and scalably connect the first part 132A of the first tube connector 606 to the second part 134B of the first tube connector 606, such that the first channel 142A fluidly communicates with the second channel 144B to acoustically couple the first tubing 110 to the electronic module 502.

As shown in FIG. 10, the modular stethoscope 600 further includes a third tube connector 109. The third tube connector 109 is connected to each of the electronic module 502 and the second tubing 112. The third tube connector 109 includes a first part 132B (shown in FIGS. 11A and 11B). The first part 132B is substantially similar to the first part 132A (shown in FIGS. 4A and 4B), with features equivalent to those of the first part 132A, shown in FIGS. 4A and 4B, designated by counterpart reference numbers suffixed with “B” instead of “A”. However, the first part 132B includes a threaded portion 143B instead of the barbed portion 140A of the first part 132A (shown in FIGS. 4A and 4B).

Referring to FIGS. 10, 11A, and 11B, the first part 132B of the third tube connector 109 includes the threaded portion 143B configured to be threadably and detachably connected to the second end 704 of the electronic module 502. In the illustrated embodiment of FIG. 10, the first part 132B defines a first channel 142B extending therethrough, such that the first channel 142B is configured to be disposed in fluid communication with the electronic module 502. The electronic module 502 includes corresponding threads (not shown) at the second end 704 for threadably coupling with the threaded portion 143B of the first part 132B of the third tube connector 109.

The third tube connector 109 further includes the second part 134A (shown in FIGS. 5A and 5B) separate from the first part 132B. Referring to FIGS. 5A, 5B, and 10, the second part 134A of the third tube connector 109 includes the barbed portion 141A configured to be at least partially received within the second tubing 112 to connect the second part 134A to the second tubing 112. The second part 134A defines the second channel 144A extending therethrough, such that the second channel 144A is configured to be disposed in fluid communication with the second tubing 112. The third tube connector 109 further includes the quick coupling member 146A configured to detachably and scalably connect the first part 132B of the third tube connector 109 to the second part 134A of the third tube connector 109, such that the first channel 142B fluidly communicates with the second channel 144A to acoustically couple the electronic module 502 to the second tubing 112.

As shown in FIGS. 12A and 12B, in some embodiments, the electronic module 502 is threadably and detachably connected to the second part 134B of the first tube connector 606 at the first end 702. Furthermore, the electronic module 502 is threadably and detachably connected to the first part 132B of the third tube connector 109 at the second end 704.

The first part 132B and the second part 134B may be part of other tube connectors (in addition to the third and first tube connectors 109, 606) used in different configurations of modular stethoscopes of the present disclosure. The reference numerals associated with the first and second parts 132B, 134B may be reused or counterpart reference numerals may be used when describing the other tube connectors.

FIG. 13 illustrates a block diagram depicting components of the electronic module 502 according to an embodiment of the present disclosure. The electronic module 502 includes a core electronics unit 1700. The core electronics unit 1700 includes a processor 1704 (e.g., a digital signal processor), a memory 1706, a signal conversion circuitry 1714 (which may include an analog circuitry), and a radio system 1712. In some embodiments, the radio system 1712 may include a software defined radio (SDR) system. It may be noted that the components of the core electronics unit 1700 may be communicably and electrically coupled to each other. In some embodiments, the components of the core electronics unit 1700 may be integrated in a processor module or a system on chip (SoC) that is installable in the electronic module 502.

The electronic module 502 further includes a signal conditioning and conversion circuitry 1702. The signal conditioning and conversion circuitry 1702 may be communicably and electrically coupled to the core electronics unit 1700. The electronic module 502 further includes a power supply 1708. The power supply 1708 may incorporate a battery 1710 or a power source different from the battery 1710. The power supply 1708 and the battery 1710 may provide electrical power to components of the electronics module 502 for operation of the electronics module 502. Thus, the electronic module 502 is configured to electronically process the acoustic waves 154 to generate the processed acoustic waves 526.

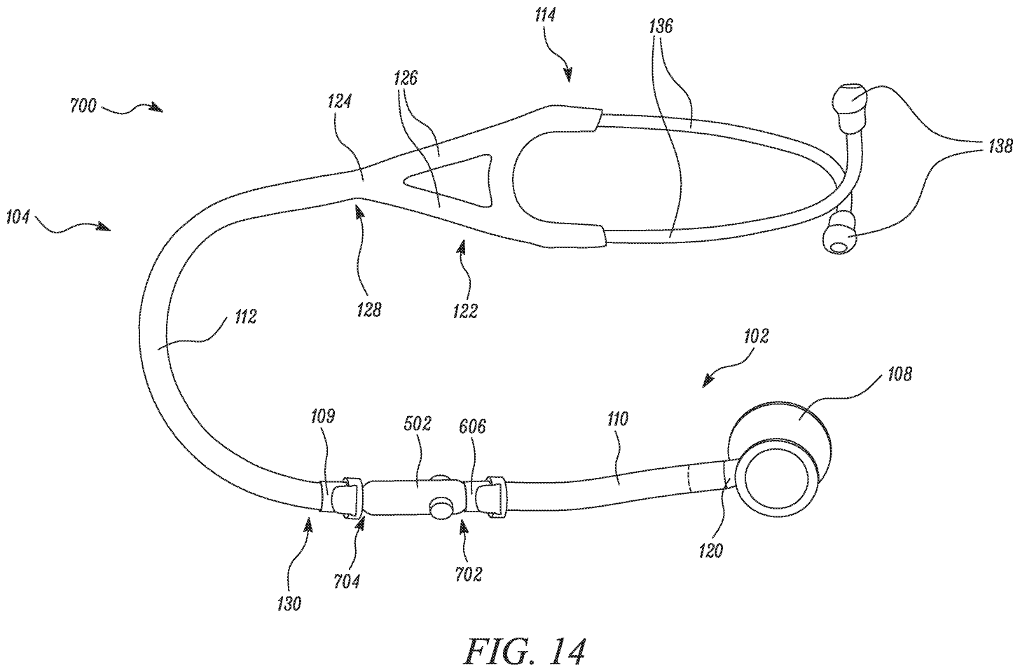

FIG. 14 illustrates a modular stethoscope 700 according to another embodiment of the present disclosure. The modular stethoscope 700 is substantially similar to the modular stethoscope 600 shown in FIG. 10. However, in the illustrated embodiment of FIG. 14, the first module 102 is disposable. Specifically, the chestpiece 108 and the first tubing 110 are disposable. Therefore, the first module 102 may be disposed of and replaced after each use. Specifically, the chestpiece 108 and the first tubing 110 may be disposed of and replaced after each use. In some cases, the user may send the first module 102 for hospital reprocessing. Therefore, the second module 104 including the headset 114 may be used with the first module 102 that is disposable to provide improved sound quality, fit, and noise isolation to the user compared to conventional disposable stethoscopes. Moreover, since only the chestpiece 108 and the first tubing 110 are disposable, while the second module 104 and the electronic module 502 are reusable, a recurring cost of the modular stethoscope 700 may be lower than conventional disposable stethoscopes. Further, disposal of the chestpiece 108 and the first tubing 110 may prevent cross-infection among patients.

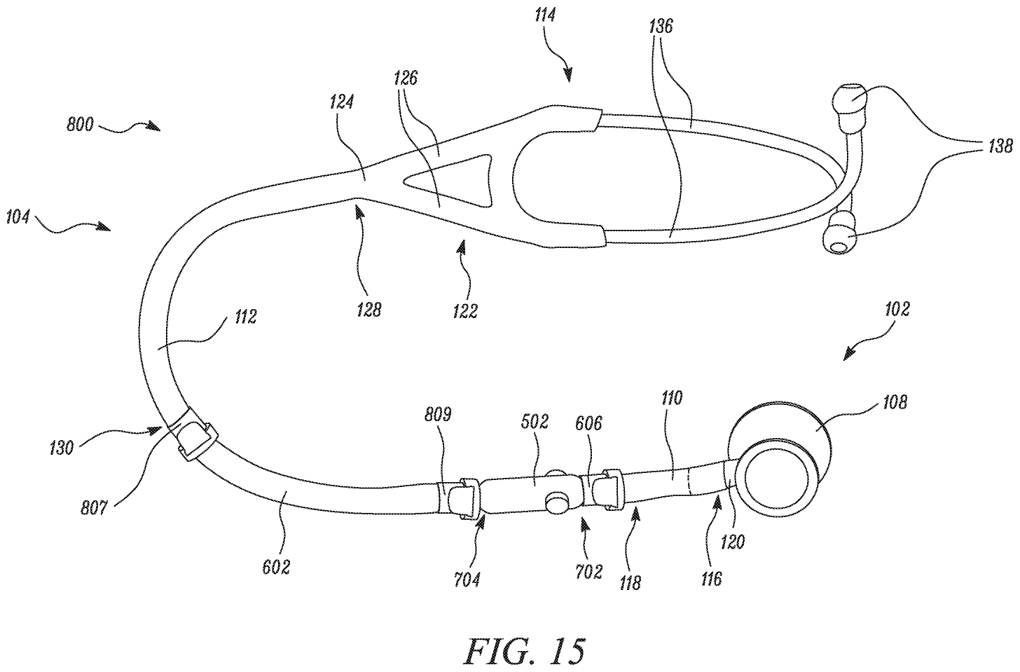

FIG. 15 illustrates a modular stethoscope 800 according to another embodiment of the present disclosure. The modular stethoscope 800 is similar to the modular stethoscope 600 shown in FIG. 10, with features equivalent to the modular stethoscope 600 designated by like reference numbers. However, in the illustrated embodiment of FIG. 15, the modular stethoscope 800 further includes an extension tubing 602 disposed between the first module 102 and the second module 104 to detachably connect the first module 102 and the second module 104. The extension tubing 602 may be disposed in fluid communication with each of the first module 102 and the second module 104. In some cases, the extension tubing 602 may be detachably connected to each of the first module 102 and the second module 104 to increase a length of the modular stethoscope 800. Increasing the length of the modular stethoscope 800 may be beneficial when a total length of the first tubing 110 and the second tubing 112 is less than a required length, depending on desired application attributes.

In the illustrated embodiment of FIG. 15, the first tube connector 606 is fluidly disposed between the first tubing 110 and the electronic module 502. Moreover, the modular stethoscope 800 further includes a third tube connector 809. The third tube connector 809 is connected to each of the electronic module 502 and the extension tubing 602.

The third tube connector 809 includes the first part 132B (shown in FIGS. 11A and 11B). Referring to FIGS. 11A, 11B, and 15, the first part 132B of the third tube connector 809 includes the threaded portion 143B configured to be threadably and detachably connected to the second end 704 of the electronic module 502. In the illustrated embodiment of FIG. 15, the first part 132B defines the first channel 142B extending therethrough, such that the first channel 142B is configured to be disposed in fluid communication with the electronic module 502.

The third tube connector 809 further includes the second part 134A (shown in FIGS. 5A and 5B) separate from the first part 132B. Referring to FIGS. 5A, 5B, and 15, the second part 134A of the third tube connector 809 includes the barbed portion 141A configured to be at least partially received within the extension tubing 602 to connect the second part 134A to the extension tubing 602. The second part 134A defines the second channel 144A extending therethrough, such that the second channel 144A is configured to be disposed in fluid communication with the extension tubing 602. The third tube connector 809 further includes the quick coupling member 146A configured to detachably and scalably connect the first part 132B (shown in FIGS. 11A and 11B) of the third tube connector 809 to the second part 134A of the third tube connector 809, such that the first channel 142B fluidly communicates with the second channel 144A to acoustically couple the electronic module 502 to the extension tubing 602.

In some embodiments, the modular stethoscope 800 further includes a second tube connector 807 fluidly disposed between the extension tubing 602 and the second tubing 112. Referring to FIGS. 4A-6B and 15, the second tube connector 807 includes the first part 132A, the second part 134A separate from the first part 132A, and the quick coupling member 146A. The quick coupling member 146A is configured to detachably and scalably connect the first part 132A of the second tube connector 807 to the second part 134A of the second tube connector 807.

The first part 132A of the second tube connector 807 includes the barbed portion 140A configured to be at least partially received within the second tubing 112 to connect the first part 132A to the extension tubing 602. The first part 132A defines the first channel 142A extending therethrough, such that the first channel 142A is configured to be disposed in fluid communication with the extension tubing 602.

The second part 134A of the second tube connector 807 includes the barbed portion 141A configured to be at least partially received within the second tubing 112 to connect the second part 134A to the second tubing 112. The second part 134A defines the second channel 144A extending therethrough, such that the second channel 144A is configured to be disposed in fluid communication with the second tubing 112.

The quick coupling member 146A of the second tube connector 807 is configured to detachably and sealably connect the first part 132A of the second tube connector 807 to the second part 134A of the second tube connector 807, such that the first channel 142A fluidly communicates with the second channel 144A to acoustically couple the extension tubing 602 to the second tubing 112.

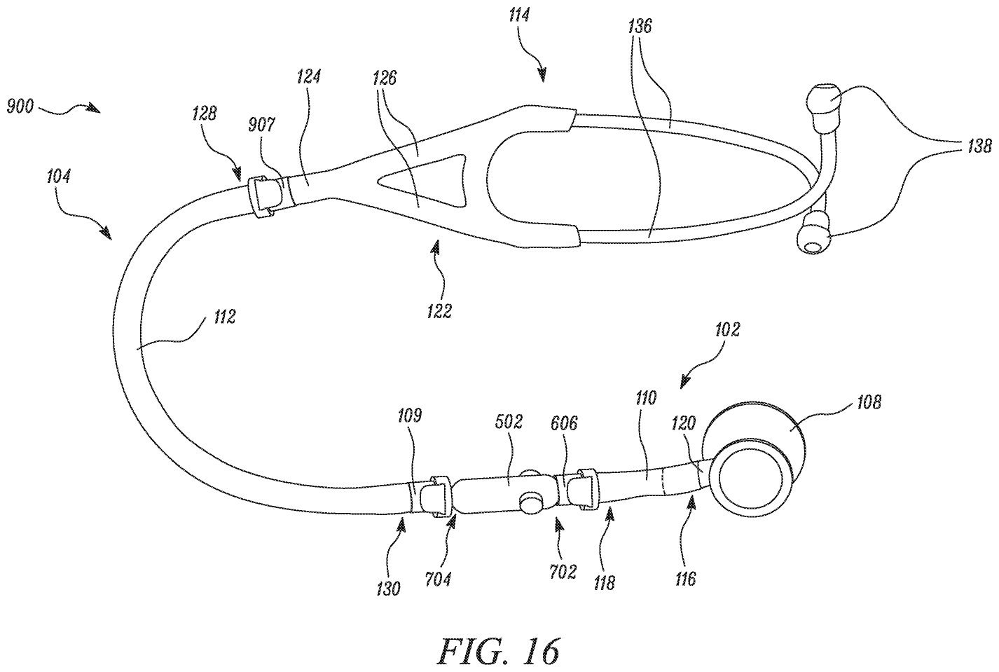

FIG. 16 illustrates a modular stethoscope 900 according to another embodiment of the present disclosure. The modular stethoscope 900 is substantially similar to the modular stethoscope 600 shown in FIG. 10. However, in the illustrated embodiment of FIG. 16, the yoke 122 is formed separately from the second tubing 112, such that the inlet tube 124 is separate from the second tubing 112. In other words, the inlet tube 124 is not integral with the second tubing 112. Moreover, the second proximal end 128 of the second tubing 112 is adjacent to the inlet tube 124 of the yoke 122.

Therefore, the modular stethoscope 900 further includes a second tube connector 907. The second tube connector 907 of the modular stethoscope 900 is substantially similar to the second tube connector 107 of the modular stethoscope 500 of FIG. 9.

Specifically, the second tube connector 907 is fluidly disposed between the second tubing 112 and the headset 114. The second tube connector 907 detachably and fluidly connects the second tubing 112 to the inlet tube 124 of the headset 114. In other words, the second tube connector 907 detachably and fluidly connects the second tubing 112 to the inlet tube 124 of the yoke 122.

Referring to FIGS. 2A-6B and 16, the second tube connector 907 includes the first part 132A, the second part 134 separate from the first part 132A, and the quick coupling member 146A. The quick coupling member 146A is configured to detachably and scalably connect the first part 132A of the second tube connector 907 to the second part 134A of the second tube connector 907.

The first part 132A of the second tube connector 907 includes the barbed portion 140A configured to be at least partially received within the second tubing 112 to connect the first part 132A to the second tubing 112. The first part 132A defines the first channel 142A extending therethrough, such that the first channel 142A is configured to be disposed in fluid communication with the second tubing 112.

The second part 134A of the second tube connector 907 includes the barbed portion 141A configured to be at least partially received within the inlet tube 124 of the headset 114 to connect the second part 134A to the inlet tube 124. In other words, the second part 134A includes the barbed portion 141A configured to be at least partially received within the inlet tube 124 of the yoke 122 to connect the second part 134A to the inlet tube 124. The second part 134A defines the second channel 144A extending therethrough, such that the second channel 144A is configured to be disposed in fluid communication with the inlet tube 124.

The quick coupling member 146A of the second tube connector 907 is configured to detachably and sealably connect the first part 132A to the second part 134A, such that the first channel 142A fluidly communicates with the second channel 144A to acoustically couple the second tubing 112 to the inlet tube 124 of the headset 114. In other words, the quick coupling member 146A is configured to detachably and sealably connect the first part 132A to the second part 134A, such that the first channel 142A fluidly communicates with the second channel 144A to acoustically couple the second tubing 112 to the inlet tube 124 of the yoke 122.

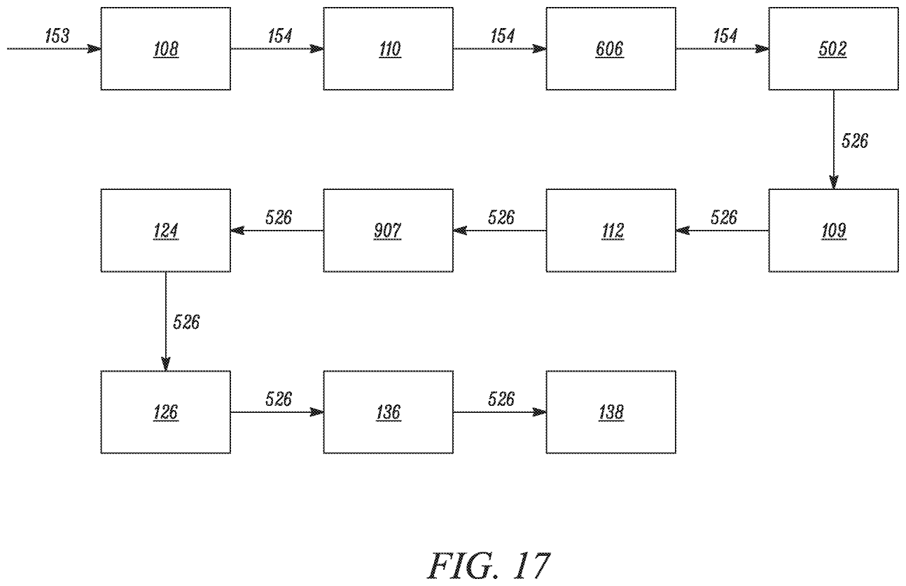

FIG. 17 illustrates a block diagram depicting a transmission of the acoustic waves 154 in the modular stethoscope 900 of FIG. 16 according to an embodiment of the present disclosure. Referring to FIGS. 16 and 17, the chestpiece 108 receives the sound 153 from the patient. The sound 153 may be from the region of the body of the patient undergoing auscultation by the user. In some embodiments, the chestpiece 108 transmits the sound 153 as the acoustic waves 154. As discussed above, the chestpiece 108 is configured to transmit the acoustic waves 154 through the first tubing 110.

Since the first tube connector 606 is fluidly disposed between the first tubing 110 of the first module 102 and the electronic module 502, the acoustic waves 154 are transmitted through the first tube connector 606 to the electronic module 502. The electronic module 502 is configured to electronically process the acoustic waves 154 to generate the processed acoustic waves 526. As discussed above, the electronic module 502 is fluidly disposed between the first tube connector 606 and the third tube connector 109. Therefore, the processed acoustic waves 526 are transmitted through the third tube connector 109 to the second tubing 112 of the second module 104.

Further, the processed acoustic waves 526 are transmitted through the second tube connector 907. In the illustrated embodiment of FIG. 16, the second tube connector 907 is fluidly disposed between the second tubing 112 and the inlet tube 124 of the yoke 122. Further, the headset 114 includes the yoke 122 including the inlet tube 124 disposed proximal to and in fluid communication with the second tubing 112. The yoke 122 further includes a pair of outlet tubes 126 disposed in fluid communication with the inlet tube 124. Moreover, the headset 114 further includes the pair of ear tubes 136. Each of the pair of ear tubes 136 is connected to the corresponding outlet tube 126 of the pair of outlet tubes 126. Therefore, the processed acoustic waves 526 are further transmitted through the inlet tube 124 to the pair of outlet tubes 126. The processed acoustic waves 526 are further transmitted through the pair of ear tubes 136, and finally to the pair of earpieces 138. The user may receive the sound 153 as the processed acoustic waves 526 through the pair of earpieces 138.

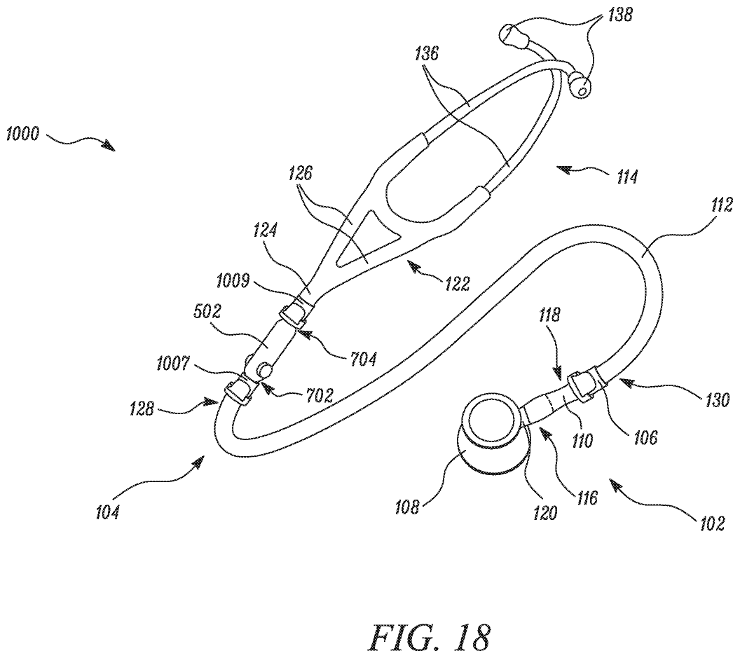

FIG. 18 illustrates a modular stethoscope 1000 according to another embodiment of the present disclosure. The modular stethoscope 1000 includes the first module 102 and the second module 104. In the illustrated embodiment of FIG. 18, the modular stethoscope 1000 further includes the electronic module 502 fluidly disposed between the second tubing 112 and the inlet tube 124 of the yoke 122. The electronic module 502 is configured to receive the acoustic waves 154 (shown in FIG. 13) from the second tubing 112 and electronically process the acoustic waves 154 to generate the processed acoustic waves 526 (shown in FIG. 13), such that the inlet tube 124 of the yoke 122 receives the processed acoustic waves 526 from the electronic module 502.

Further, the electronic module 502 includes the first end 702 proximal to the second tubing 112, and the second end 704 opposite to the first end 702 and distal to the second tubing 112. In some embodiments, the second end 704 is proximal to the inlet tube 124 of the headset 114. In other words, the second end 704 is proximal to the inlet tube 124 of the yoke 122.

The tube connector 106 detachably connected to the first tubing 110 includes the first tube connector 106. In the illustrated embodiment of FIG. 18, the first tube connector 106 is fluidly disposed between the first tubing 110 of the first module 102 and the second tubing 112 of the second module 104. Further, the first tube connector 106 acoustically couples the first tubing 110 to the second tubing 112.

The modular stethoscope 1000 further includes a second tube connector 1007 detachably connecting the second tubing 112 to the electronic module 502, and a third tube connector 1009 detachably connecting the electronic module 502 to the inlet tube 124 of the yoke 122.

The second tube connector 1007 includes the first part 132A (shown in FIGS. 4A and 4B). Referring to FIGS. 4A, 4B, and 18, the second tube connector 1007 includes the first part 132A including the barbed portion 140A configured to be at least partially received within the second tubing 112 to connect the first part 132A to the second tubing 112. The first part 132A defines the first channel 142A extending therethrough, such that the first channel 142A is configured to be disposed in fluid communication with the second tubing 112.

The second tube connector 1007 further includes the second part 134B (shown in FIGS. 11C and 11D). Referring to FIGS. 11C, 11D, and 18, the second tube connector 1007 further includes the second part 134B separate from the first part 132A. The second part 134B includes the threaded portion 145B configured to be threadably and detachably connected to the first end 702 of the electronic module 502. The second part 134B defines the second channel 144B extending therethrough, such that the second channel 144B is configured to be disposed in fluid communication with the electronic module 502.

The second tube connector 1007 further includes the quick coupling member 146B. Referring to FIGS. 4A, 4B, 11C, 11D, and 18, the second tube connector 1007 includes the quick coupling member 146B configured to detachably and scalably connect the first part 132A of the second tube connector 1007 to the second part 134B of the second tube connector 1007, such that the first channel 142A fluidly communicates with the second channel 144B to acoustically couple the second tubing 112 to the electronic module 502.

Moreover, the third tube connector 1009 includes the first part 132B (shown in FIGS. 11A and 11B) including the threaded portion 143B configured to be threadably and detachably connected to the second end 704 of the electronic module 502. The first part 132B defines the first channel 142B extending therethrough, such that the first channel 142B is configured to be disposed in fluid communication with the electronic module 502.

The third tube connector 1009 further includes the second part 134A (shown in FIGS. 5A and 5B) separate from the first part 132B. The second part 134A includes the barbed portion 141A configured to be at least partially received within the inlet tube 124 of the yoke 122 to connect the second part 134A to the inlet tube 124. The second part 134A defines the second channel 144A extending therethrough, such that the second channel 144A is configured to be disposed in fluid communication with the inlet tube 124.

The third tube connector 1009 further includes the quick coupling member 146A configured to detachably and scalably connect the first part 132B of the third tube connector 1009 to the second part 134A of the third tube connector 1009, such that the first channel 142B fluidly communicates with the second channel 144A to acoustically couple the electronic module 502 to the inlet tube 124.

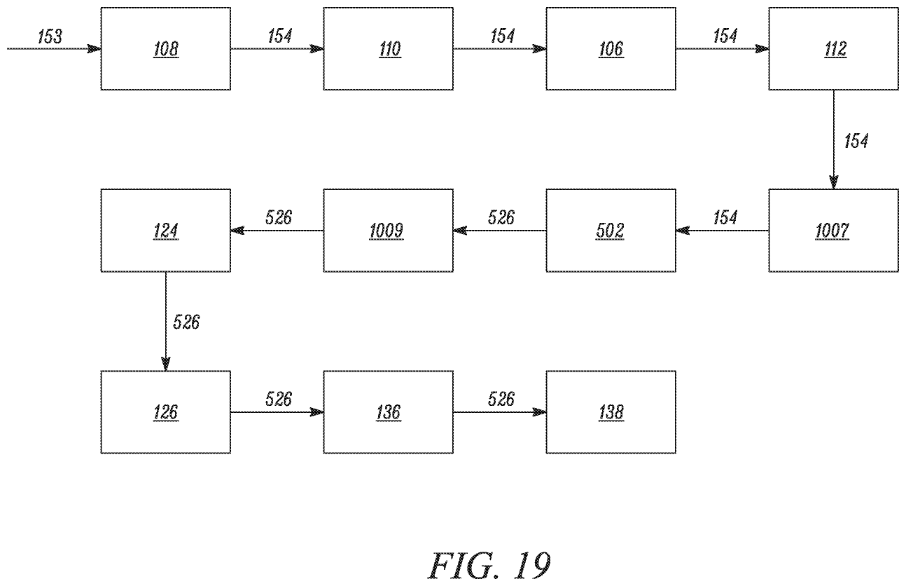

FIG. 19 illustrates a block diagram depicting a transmission of the acoustic waves 154 in the modular stethoscope 1000 of FIG. 18 according to an embodiment of the present disclosure. Referring to FIGS. 18 and 19, the chestpiece 108 receives the sound 153 from the patient. The sound 153 may be from the region of the body of the patient undergoing auscultation by the user. In some embodiments, the chestpiece 108 transmits the sound 153 as the acoustic waves 154. As discussed above, the chestpiece 108 is configured to transmit the acoustic waves 154 through the first tubing 110.

Since the first tube connector 106 is fluidly disposed between the first tubing 110 of the first module 102 and the second tubing 112 of the second module 104, the acoustic waves 154 are transmitted through the first tube connector 106 to the second tubing 112. Further, since the second tube connector 1007 is fluidly disposed between the second tubing 112 of the second module 104 and the electronic module 502, the acoustic waves 154 are transmitted through the second tube connector 1007 to the electronic module 502. The electronic module 502 is configured to electronically process the acoustic waves 154 to generate the processed acoustic waves 526.

The processed acoustic waves 526 are further transmitted to the third tube connector 1009. In the illustrated embodiment of FIG. 18, the third tube connector 1009 is fluidly disposed between the electronic module 502 and the inlet tube 124. Therefore, the processed acoustic waves 526 are further transmitted through the third tube connector 1009 to the inlet tube 124.

In the illustrated embodiment of FIG. 18, the headset 114 includes the yoke 122 including the inlet tube 124 disposed proximal to and in fluid communication with the second tubing 112. The yoke 122 further includes the pair of outlet tubes 126 disposed in fluid communication with the inlet tube 124. Moreover, the headset 114 further includes the pair of car tubes 136. Each of the pair of car tubes 136 is connected to the corresponding outlet tube 126 of the pair of outlet tubes 126. Therefore, the processed acoustic waves 526 are further transmitted through the inlet tube 124 to the pair of outlet tubes 126. The processed acoustic waves 526 are further transmitted through the pair of car tubes 136, and finally to the pair of earpieces 138. The user may receive the sound 153 as the processed acoustic waves 526 through the pair of earpieces 138.

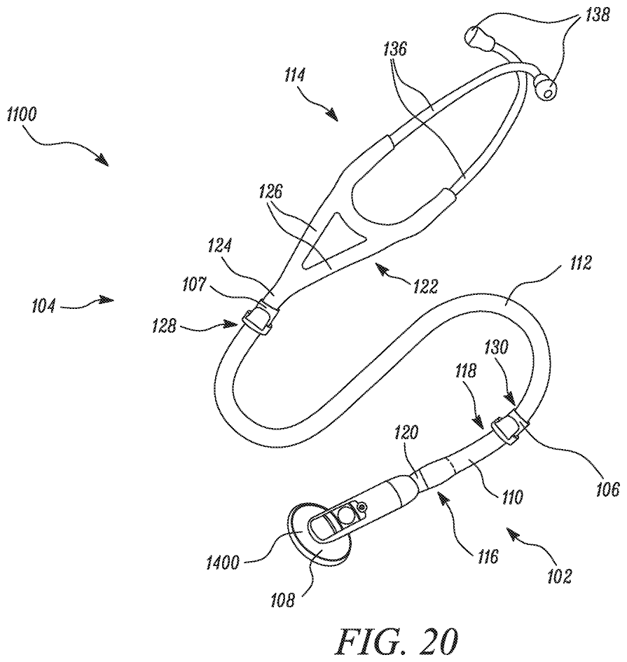

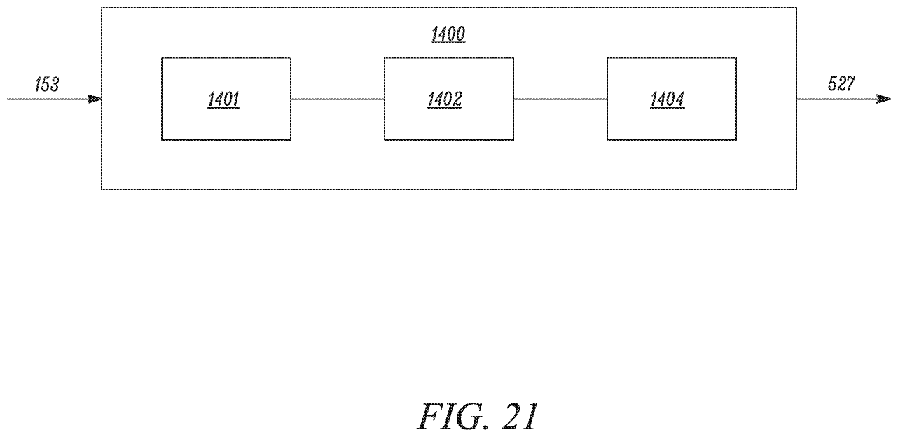

FIG. 20 illustrates a modular stethoscope 1100 according to another embodiment of the present disclosure. The modular stethoscope 1100 is substantially similar to the modular stethoscope 500 shown in FIG. 9. However, in the illustrated embodiment of FIG. 20, the chestpiece 108 is an electrical chestpiece 1400. Hereinafter, the chestpiece 108 may be interchangeably referred to as “the electrical chestpiece 1400”. The chestpiece 108 is configured to transmit the acoustic waves 154 (shown in FIG. 3A) through the first tubing 110. In other words, the electrical chestpiece 1400 is configured to transmit the acoustic waves 154 (shown in FIG. 3A) through the first tubing 110. In some embodiments, the chestpiece 108 includes an electrical transducer 1401 (shown in FIG. 21). In other words, electrical chestpiece 1400 includes the electrical transducer 1401 (shown in FIG. 21). The electrical chestpiece 1400 will be described in detail with reference to FIG. 21.

FIG. 21 illustrates a block diagram depicting components of the electrical chestpiece 1400 according to an embodiment of the present disclosure. The electrical chestpiece 1400 may be placed on the region of the body of the patient requiring auscultation by the user. The electrical chestpiece 1400 receives the sound 153 from the patient. Further, the electrical chestpiece 1400 electrically processes the sound 153 generate processed acoustic waves 527.

In the illustrated embodiment of FIG. 21, the chestpiece 108 includes the electrical transducer 1401. In other words, the electrical chestpiece 1400 includes the electrical transducer 1401. In some embodiments, the electrical chestpiece 1400 may be configured to modulate or generate a medical measurement signal in response to deformation of the electric transducer 1401. Suitable transducers may incorporate piezoelectric material (organic and/or inorganic piezoelectric material), such as piezoelectric film, piezoresistive material, strain gauges, capacitive or inductive elements, a linear variable differential transformer, and other materials or elements that modulate or generate an electrical signal in response to deformation. Suitable piezo materials may include polymer films, polymer foams, ceramic, composite materials, and combinations thereof. The electrical chestpiece 1400 may incorporate arrays of electrical transducers of the same or different transducer type and/or different transducer materials, all of which may be connected in series, individually, or in a multi-layered structure.

In the illustrated embodiment of FIG. 21, the electrical chestpiece 1400 further includes a digital filter 1402. The digital filter 1402 may be configured to provide digital filtering to the processed medical measurement signals and generate filtered signals. In some embodiments, the digital filter 1402 may be configured to provide one or more filters. Each filter may be operable to provide a transfer function resulting in a frequency response similar to that of a mechanical stethoscope (for example, the modular stethoscope 100 of FIG. 1). A frequency response of the electrical chestpiece 1400 may depend on a design of a signal circuitry and a filter used in the digital filter 1402. In some embodiments, each filter is operable to provide a transfer function resulting in a frequency response of the electrical chestpiece 1400 that is close to the frequency response of a selected mechanical stethoscope within a predetermined threshold. In some cases, the predetermined threshold may be a threshold that is perceptually relevant to users. For example, the predetermined threshold can be a 2 decibel (dB) deviation for a frequency range of about 10 hertz (Hz) to about 3000 Hz.

Moreover, in the illustrated embodiment of FIG. 21, the electrical chestpiece 1400 further includes a signal circuit 1404. The signal circuit 1404 is configured to receive the medical measurement signals generated by the electrical chestpiece 1400. In some embodiments, the medical measurement signals are analog signals and the signal circuit 1404 is configured to convert the medical measurement signals to digital medical measurement signals. In some implementations, the signal circuit 1404 may be further configured to provide analog amplification and/or analog filtering to the medical measurement signals before the analog-to-digital conversion. The signal circuit 1404 may precondition medical measurement signals based on the desired characteristics of the medical measurement signals.

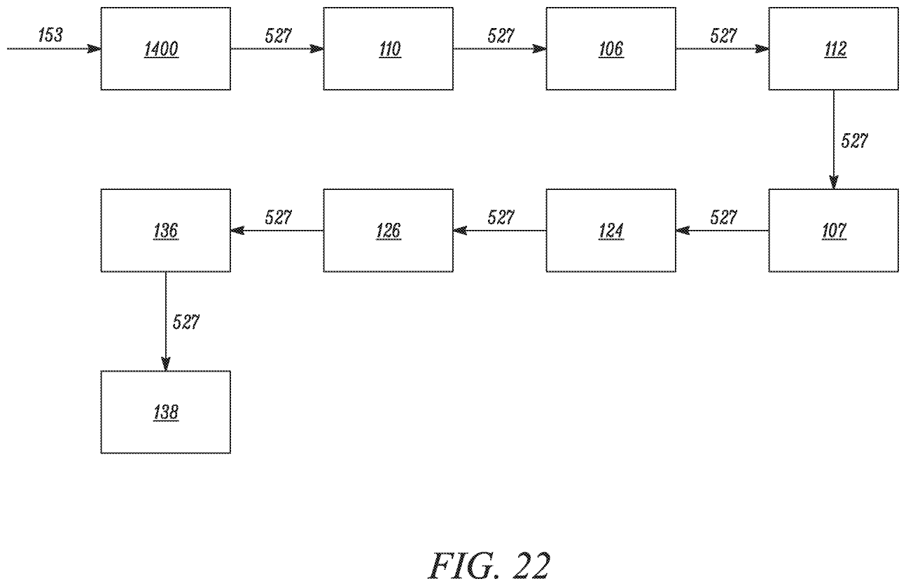

FIG. 22 illustrates a block diagram depicting a transmission of the acoustic waves 154 in the modular stethoscope 1100 of FIG. 20 according to an embodiment of the present disclosure. Referring to FIGS. 20 and 22, the electrical chestpiece 1400 transmits the sound 153 as the acoustic waves 154 from the patient. In some embodiments, the electrical chestpiece 1400 transmits the acoustic waves 154 as the processed acoustic waves 527. Therefore, the acoustic waves 154 are transmitted from the electrical chestpiece 1400, and through the first tubing 110 as the processed acoustic waves 527.

Since the first tube connector 106 is fluidly disposed between the first tubing 110 of the first module 102 and the second tubing 112 of the second module 104, the processed acoustic waves 527 are transmitted through the first tube connector 106 to the second tubing 112. Further, since the second tube connector 107 is fluidly disposed between the second tubing 112 of the second module 104 and the inlet tube 124, the processed acoustic waves 527 are transmitted through the second tube connector 514 to the inlet tube 124.

In the illustrated embodiment of FIG. 20, the headset 114 includes the yoke 122 including the inlet tube 124 disposed proximal to and in fluid communication with the second tubing 112. The yoke 122 further includes the pair of outlet tubes 126 disposed in fluid communication with the inlet tube 124. Moreover, the headset 114 further includes the pair of car tubes 136. Each of the pair of car tubes 136 is connected to the corresponding outlet tube 126 of the pair of outlet tubes 126. Therefore, the processed acoustic waves 527 are further transmitted through the inlet tube 124 to the pair of outlet tubes 126. The processed acoustic waves 527 are further transmitted through the pair of car tubes 136, and finally to the pair of earpieces 138. The user may receive the sound 153 as the processed acoustic waves 527 through the pair of earpieces 138.

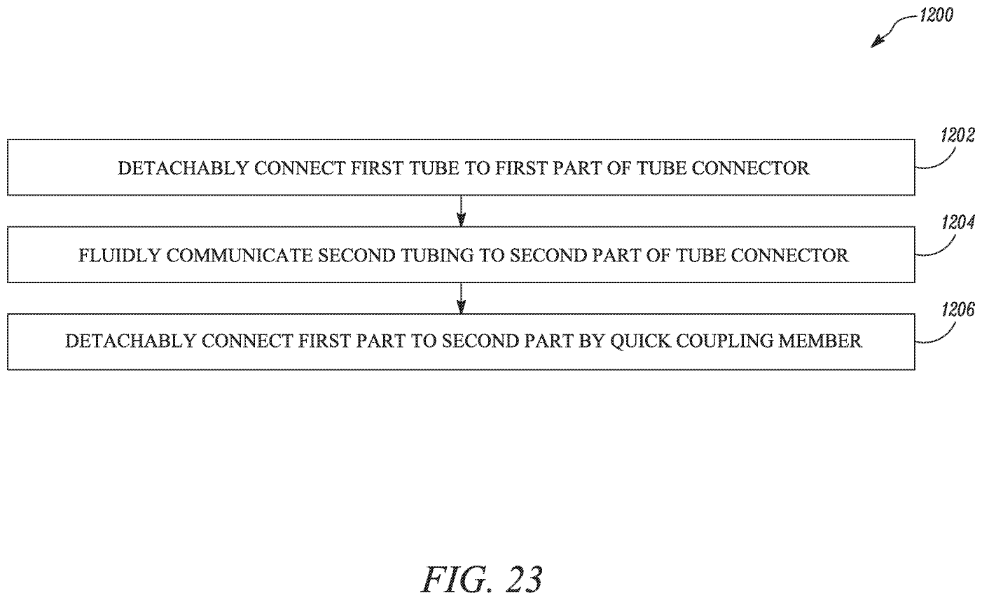

The present disclosure further provides a method 1200 of using the modular stethoscopes 100, 400, 500, 600, 700, 800, 900, 1000, 1100 of the present disclosure. The method 1200 will be described with reference to FIGS. 1-20 and 23. The method 1200 includes the following steps:

At step 1202, the method 1200 includes connecting the first tubing 110 to the first part 132A of the tube connector 106. As shown in FIGS. 2A and 2B, in some embodiments, connecting the first tubing 110 to the first part 132A of the tube connector 106 further includes at least partially inserting the barbed portion 140A of the first part 132A within the first tubing 110.

At step 1204, the method 1200 further includes fluidly communicating the second tubing 112 to the second part 134A of the tube connector 106. In some embodiments, fluidly communicating the second tubing 112 to the second part 134A further includes connecting the second tubing 112 to the second part 134A. As shown in FIGS. 2A and 2B, in some embodiments, connecting the second tubing 112 to the second part 134A further includes at least partially inserting the barbed portion 141A of the second part 134A within the second tubing 112.

As shown in FIGS. 10-12B, in some embodiments, fluidly communicating the second tubing 112 to the second part 134B further includes detachably connecting the second part 134B of the tube connector 606 to the electronic module 502, such that the electronic module 502 is fluidly disposed between the first tubing 110 and the second tubing 112. Moreover, as shown in FIG. 18, in some embodiments, fluidly communicating the second tubing 112 to the second part 134B further includes detachably connecting the electronic module 502 to the second tubing 112 by the second tube connector 1007.

At step 1206, the method 1200 further includes detachably connecting the first part 132A to the second part 134A by the quick coupling member 146A.

As shown in FIG. 9, in some embodiments, the method 1200 further includes connecting the second tubing 112 to the headset 114 by the second tube connector 107.

As shown in FIG. 18, in some embodiments, the method 1200 further includes detachably connecting the electronic module 502 to the second tubing 112 by the second tube connector 1007. Furthermore, in some embodiments, the method 1200 further includes detachably connecting the headset 114 to the electronic module 502 by the third tube connector 1009.

In some embodiments, the method 1200 further includes detachably connecting the chestpiece 108 to the first tubing 110. In some other embodiments, the method 1200 further includes detachably connecting the electrical chestpiece 1400 to the first tubing 110.

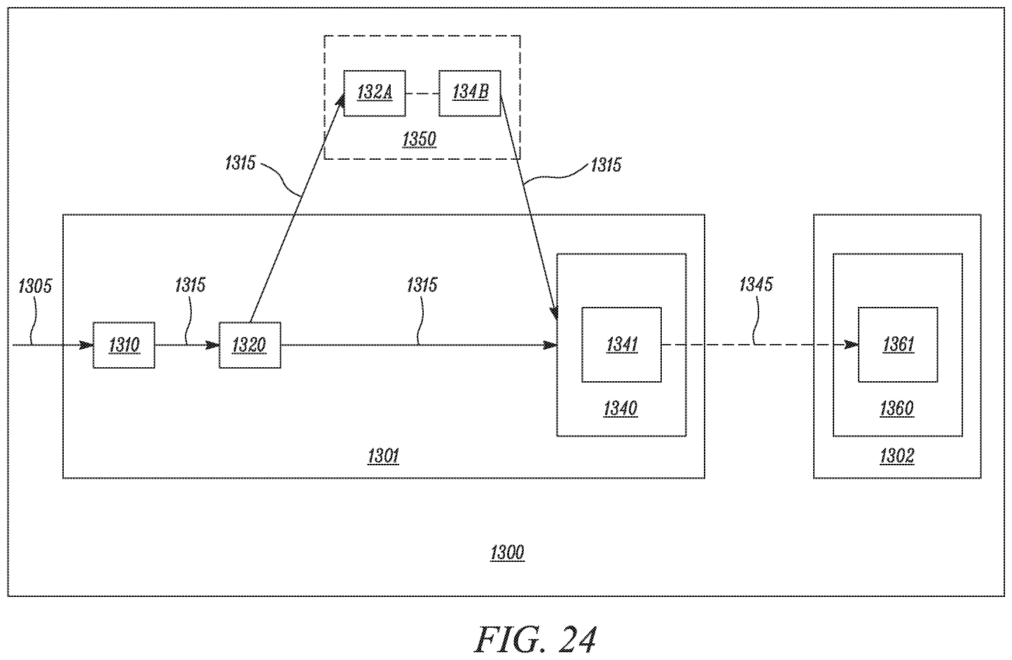

FIG. 24 illustrates a schematic block diagram of a modular stethoscope 1300 according to another embodiment of the present disclosure.

The modular stethoscope 1300 includes a first module 1301 including a chestpiece 1310 and a tubing 1320 disposed in fluid communication with and connected to the chestpiece 1310. The chestpiece 1310 is configured to transmit acoustic waves 1315 through the tubing 1320. Specifically, the chestpiece 1310 may be configured to receive a sound 1305 and transmit the sound 1305 as the acoustic waves 1315 through the tubing 1320. The chestpiece 1310 may be substantially similar to the chestpiece 108 (shown in FIG. 1). Further, the tubing 1320 may be substantially similar to the first tubing 110 (shown in FIG. 1). In some embodiments, the chestpiece 1310 may be a mechanical chestpiece substantially similar to the chestpiece 108 shown in FIG. 7. In some other embodiments, the chestpiece 1310 may be an electrical chestpiece similar to the electrical chestpiece 1400 shown in FIG. 21.

The first module 1301 further includes an electronic module 1340 detachably connected to and disposed in fluid communication with the tubing 1320. The electronic module 1340 may be substantially similar to the electronic module 502 shown in FIG. 10. The electronic module 1340 is configured to receive the acoustic waves 1315 from the tubing 1320 and electronically process the acoustic waves 1315 to generate processed acoustic waves 1345. However, in some embodiments, where the chestpiece 1310 is the electrical chestpiece, the electronic module 1340 may receive first processed acoustic waves from the electrical chestpiece, and process the first processed acoustic waves to generate the processed acoustic waves 1345. In some embodiments, the electronic module 1340 includes a transmitter 1341. The transmitter 1341 may allow the electronic module 1340 to wirelessly transmit the processed acoustic waves 1345 to other electronic devices.

Referring to FIGS. 4A-6B, 11C, and 11D, in some embodiments, the modular stethoscope 1300 further includes a tube connector 1350 fluidly disposed between the tubing 1320 and the electronic module 502. In some embodiments, the tube connector 1350 includes the first part 132A including the barbed portion 140A configured to be at least partially received within the tubing 1320 to connect the first part 132A to the tubing 1320. The first part 132A defines the first channel 142A extending therethrough, such that the first channel 142A is configured to be disposed in fluid communication with the tubing 1320. In some embodiments, the tube connector 1350 further includes the second part 134B separate from the first part 132A. The second part 134B includes the threaded portion 145B configured to be threadably and detachably connected to an end of the electronic module 1340. The second part 134B defines the second channel 144B extending therethrough, such that the second channel 144B is configured to be disposed in fluid communication with the electronic module 1340. In some embodiments, the tube connector 1350 further includes the quick coupling member 146B configured to detachably and scalably connect the first part 132A of the tube connector 1350 to the second part 134B of the tube connector 1350, such that the first channel 142A fluidly communicates with the second channel 144B to acoustically couple the tubing 1320 to the electronic module 1340.

The modular stethoscope 1300 further includes a second module 1302 including a headset 1360 disposed in wireless communication with the electronic module 1340. The headset 1360 is configured to receive the processed acoustic waves 1345 from the electronic module 1340. Specifically, in some embodiments, the headset 1360 includes a receiver 1361. The receiver 1361 may allow the headset 1360 to wirelessly receive digital signals from other electronic devices.

In some embodiments, the transmitter 1341 is configured to wirelessly transmit the processed acoustic waves 1345 to the headset 1360. Further, in some embodiments, the receiver 1361 is configured to wirelessly receive the processed acoustic waves 1345 from the electronic module 1340. In other words, the processed acoustic waves 1345 may be transmitted to the headset 1360 via wireless communication between the transmitter 1341 of the electronic module 1340 and the receiver 1361 of the headset 1360. Furthermore, the user may receive the sound 1305 as the processed acoustic waves 1345 through the headset 1360.

The modular stethoscope 1300 may be ideally suited for use underneath the personal protective equipment designed to protect the user when working in the isolation space. Specifically, the headset 1360 may remain underneath the personal protective equipment. Therefore, the headset 1360 may remain effectively clean, and may be used with a non-infectious patient by wirelessly coupling the headset 1360 with the electronic module 1340 of different first modules 1301. Therefore, the modular stethoscope 1300 may provide improved protection to the user as it may not compromise a performance of the personal protective equipment worn by the user.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A modular stethoscope comprising:
a first module comprising a chestpiece and a first tubing disposed in fluid communication with and connected to the chestpiece, wherein the chestpiece is configured to transmit acoustic waves through the first tubing;
a second module detachably connected to the first module, the second module comprising a second tubing and a headset disposed in fluid communication with the second tubing; and
a tube connector fluidly disposed between the first tubing of the first module and the second tubing of the second module, wherein the tube connector is detachably connected to the first tubing of the first module, the tube connector comprising:
a first part comprising a barbed portion configured to be at least partially received within the first tubing to connect the first part to the first tubing, wherein the first part defines a first channel extending therethrough, such that the first channel is configured to be disposed in fluid communication with the first tubing;
a second part separate from the first part, wherein the second part defines a second channel extending therethrough, such that the second channel is configured to be disposed in fluid communication with the second tubing; and
a quick coupling member configured to detachably and sealably connect the first part to the second part, such that the first channel fluidly communicates with the second channel to acoustically couple the first tubing to the second tubing.

2. The modular stethoscope of claim 1, wherein the first tubing comprises a first proximal end connected to the chestpiece, a first distal end opposite to the first proximal end and distal to the chestpiece, a first outer surface, and a first inner surface, wherein each of the first outer surface and the first inner surface extends between the first proximal end and the first distal end, wherein the first inner surface defines a first inner volume configured to transport the acoustic waves, and wherein the barbed portion of the first part of the tube connector is configured to be at least partially received within the first inner surface of the first tubing at the first distal end.

3. The modular stethoscope of claim 1, wherein the headset comprises a yoke comprising an inlet tube disposed proximal to and in fluid communication with the second tubing, and a pair of outlet tubes disposed in fluid communication with the inlet tube and disposed distal to the second tubing.

4. The modular stethoscope of claim 3, wherein the yoke is integrally formed with the second tubing, such that the inlet tube is integral with the second tubing.

5. The modular stethoscope of claim 3, wherein the headset further comprises a pair of ear tubes and a pair of earpieces, wherein each of the pair of ear tubes is connected to a corresponding outlet tube of the pair of outlet tubes of the yoke, and wherein each of the pair of earpieces is connected to a corresponding ear tube of the pair of ear tubes.

6. The modular stethoscope of claim 3, wherein the tube connector detachably connected to the first tubing comprises a first tube connector, wherein the modular stethoscope further comprises a second tube connector fluidly disposed between the second tubing and the headset, the second tube connector comprising:
a first part comprising a barbed portion configured to be at least partially received within the second tubing to connect the first part to the second tubing, wherein the first part defines a first channel extending therethrough, such that the first channel is configured to be disposed in fluid communication with the second tubing;
a second part separate from the first part, the second part comprising a barbed portion configured to be at least partially received within the inlet tube of the headset to connect the second part to the inlet tube, wherein the second part defines a second channel extending therethrough, such that the second channel is configured to be disposed in fluid communication with the inlet tube; and
a quick coupling member configured to detachably and sealably connect the first part of the second tube connector to the second part of the second tube connector, such that the first channel fluidly communicates with the second channel to acoustically couple the second tubing to the inlet tube of the headset.

7. The modular stethoscope of claim 6, wherein a ratio of a length of the second tubing to a length of the inlet tube of the yoke is at least 3.

8. The modular stethoscope of claim 6, wherein a length of the second tubing is less than or equal to a length of the inlet tube of the yoke.

9. The modular stethoscope of claim 3, further comprising an electronic module fluidly disposed between the second tubing and the inlet tube of the yoke, wherein the electronic module is configured to receive the acoustic waves from the second tubing and electronically process the acoustic waves to generate processed acoustic waves, such that the inlet tube of the yoke receives the processed acoustic waves from the electronic module, wherein the electronic module comprises a first end proximal to the second tubing, and a second end opposite to the first end and distal to the second tubing.

10. The modular stethoscope of claim 9, wherein the tube connector detachably connected to the first tubing comprises a first tube connector, and wherein the modular stethoscope further comprises a second tube connector detachably connecting the second tubing to the electronic module, and a third tube connector detachably connecting the electronic module to the inlet tube of the yoke.

11. The modular stethoscope of claim 10, wherein the second tube connector comprises:
a first part comprising a barbed portion configured to be at least partially received within the second tubing to connect the first part to the second tubing, wherein the first part defines a first channel extending therethrough, such that the first channel is configured to be disposed in fluid communication with the second tubing;
a second part separate from the first part, the second part comprising a threaded portion configured to be threadably and detachably connected to the first end of the electronic module, wherein the second part defines a second channel extending therethrough, such that the second channel is configured to be disposed in fluid communication with the electronic module; and
a quick coupling member configured to detachably and sealably connect the first part of the second tube connector to the second part of the second tube connector, such that the first channel fluidly communicates with the second channel to acoustically couple the second tubing to the electronic module.

12. The modular stethoscope of claim 10, wherein the third tube connector comprises:
a first part comprising a threaded portion configured to be threadably and detachably connected to the second end of the electronic module, wherein the first part defines a first channel extending therethrough, such that the first channel is configured to be disposed in fluid communication with the electronic module;
a second part separate from the first part, the second part comprising a barbed portion configured to be at least partially received within the inlet tube of the yoke to connect the second part to the inlet tube, wherein the second part defines a second channel extending therethrough, such that the second channel is configured to be disposed in fluid communication with the inlet tube; and
a quick coupling member configured to detachably and sealably connect the first part of the third tube connector to the second part of the third tube connector, such that the first channel fluidly communicates with the second channel to acoustically couple the electronic module to the inlet tube.

13. The modular stethoscope of claim 1, wherein the second tubing comprises a second proximal end connected to the headset, a second distal end opposite to the second proximal end and distal to the headset, a second outer surface, and a second inner surface, wherein each of the second outer surface and the second inner surface extends between the second proximal end and the second distal end, wherein the second inner surface defines a second inner volume configured to transport the acoustic waves.

14. The modular stethoscope of claim 13, wherein the second part of the tube connector comprises a barbed portion configured to be at least partially received within the second inner surface of the second tubing at the second distal end, such that the second part is connected to the second tubing.

15. The modular stethoscope of claim 1, further comprising an electronic module fluidly disposed between the first module and the second module, wherein the electronic module is configured to receive the acoustic waves from the first tubing and electronically process the acoustic waves to generate processed acoustic waves, such that the second tubing receives the processed acoustic waves from the electronic module, wherein the electronic module comprises a first end proximal to the first module, and a second end opposite to the first end and distal to the first module.

16-38. (canceled)
